(19) United States
(12) Patent Application Publication
(10) Pub. No.: US 2003/0194456 A1
(43) Pub. Date: Oct. 16, 2003

(54) Herbal composition for treating various disorders including psoriasis, a process for preparation thereof and method for treatment of such disorders

(75) Inventors: Sudershan Kumar Arora, Maharashtra (IN); Lavleen Kumar Gupta, Maharashtra (IN); Vandita Srivastava, Maharashtra (IN); Narender Sanganabhatla, Maharashtra (IN); Dinesh Balakrishna Saraf, Maharashtra (IN)

Correspondence Address: Ladas & Parry, 26 West 61 Street, New York, NY, 10023, US

(73) Assignee: LUPIN LIMITED

(21) Appl. No.: 10/340,195

(22) Filed: Jan. 10, 2003

(30) Foreign Application Priority Data
Jan. 10, 2002 (IN) 22/MUM/2002

(51) Int. Cl. A61K 35/78

(52) U.S. Cl. 424/774; 424/779

(57) ABSTRACT

The invention provides a novel herbal composition containing the extracts of the leaves and/or stem of Argemone mexicana plant, optionally containing the extracts of the fruits of Cuminum cyminum, which exhibits useful in vitro, in vivo and interesting immunological and pharmacological activities; a process for preparation thereof; and a method of treatment of psoriasis and related immunological and biological disorders by administration of the said novel herbal composition. The useful in vitro, in vivo and interesting immunological and pharmacological activities exhibited by the extracts and fractions of the leaves and/or stem of Argemone mexicana plant include immunosuppression, lymphoproliferation inhibition, cytokine modulation such as IL-2 inhibition, IFNgamma inhibition, IL-10 induction, keratinocyte proliferation inhibition, keratolytic activity, endothelial cell proliferation inhibition, inhibition of cell adhesion molecule expression such as ICAM-1, MEST inhibition, and enzymes inhibition such as p60src Tyrosine kinase, which are known to be involved in anti-psoriatic activity. The novel herbal composition(s) is useful in the treatment of various disorders, such as psoriasis including plaque psoriasis, gutatte psoriasis, pustular psoriasis and psoriasis of the nails; dermatitis and scleroderma; eczema; inflammatory disorders and other autoimmune diseases like psoriatic arthritis, rheumatoid arthritis, Crohn's disease, multiple sclerosis, irritable bowel disease, ankylosing spondilitis, systemic lupus erythremetosus and Sjogren's syndrome; allergies like asthma and chronic obstructive pulmonary disease and is safe, well-tolerated, non-toxic, with minimal and reversible adverse reactions or side effects, and most importantly, with minimal relapse or recurrence of the disease following completion of a treatment regimen. The invention also describes the presence of phosphodiesterase (III, IV and V) inhibition and 5-Lipoxygenase inhibition in the aqueous, ethanolic or aqueous-ethanolic extracts of fruits of Cuminum cyminum plant.

### FIELD OF THE INVENTION

The present invention relates to a herbal composition comprising aqueous, ethanolic or aqueous-ethanolic extracts obtained from leaves and/or stem of Argemone mexicana plant, containing a mixture of alkaloids, flavonoids, organic acids, amino acids, sugars/glycosides and salts, which exhibit useful in vitro and in vivo immunological and pharmacological activities, hitherto not known and which provide significant reduction in the rate of Psoriasis Area and Severity Index (PASI) score with better tolerability within the range of normal permissible limit.

The present invention relates to a herbal composition comprising aqueous, ethanolic or aqueous-ethanolic extracts obtained from leaves and/or stem of Argemone mexicana plant, containing a mixture of alkaloids, flavonoids, organic acids, amino acids, sugars/glycosides and salts, optionally in combination with an aqueous, ethanolic or aqueous-ethanolic extract obtained from the fruits of Cuminum cyminum plant, which exhibit useful in vitro and in vivo immunological and pharmacological activities, hitherto not known and which provide significant reduction in the rate of PASI score with better tolerability within the range of normal permissible limit.

The present invention also relates to a herbal composition comprising fractions of the aqueous, ethanolic and aqueous-ethanolic extracts obtained from leaves and/or stem of Argemone mexicana plant, containing a mixture of alkaloids, flavonoids, organic acids, amino acids, sugars/glycosides and salts, which exhibit useful in vitro and in vivo immunological and pharmacological activities, hitherto not known .

The present invention also relates to a selective process for preparation of the extracts obtained from leaves and/or stem of Argemone mexicana plant optionally in combination with an aqueous, ethanolic or aqueous-ethanolic extract obtained from the fruits of Cuminum cyminum plant contained in the herbal composition.

The present invention also relates to a selective process for preparation of the fractions such as n-butanol soluble, methanol soluble and methanol insoluble fractions from leaves and/or stem of Argemone mexicana plant contained in the herbal composition.

The invention further relates to the extracts obtained from leaves and stem of Argemone mexicana plant and the fractions obtained from leaves and stem of Argemone mexicana plant, which exhibit immunosuppression, lymphoproliferation inhibition, cytokine modulation such as IL-2 inhibition, IFNgamma inhibition, IL-10 induction, keratinocyte proliferation inhibition, keratolytic activity, endothelial cell proliferation inhibition, inhibition of cell adhesion molecule expression such as ICAM-1, MEST inhibition, and enzymes inhibition such as p60src Tyrosine kinase , which are known to be involved in anti-psoriatic activity and the usefulness of the said extracts and fractions for the treatment and prevention of skin ailments such as psoriasis including plaque psoriasis, guttate psoriasis, pustular psoriasis and psoriasis of the nails; dermatitis and scleroderma; eczema; inflammatory disorders and other autoimmune diseases like psoriatic arthritis, rheumatoid arthritis, Crohn's disease, multiple sclerosis, irritable bowel disease, ankylosing spondilitis, systemic lupus erythremetosus and Sjogren's syndrome; allergies like asthma and chronic obstructive pulmonary disease.

The invention further relates to a method of treatment of and prevention of the abovementioned disorders, in particular psoriasis comprising administering to a mammal through the oral route or by topical application the herbal composition containing the extracts obtained from leaves and/or stem of Argemone mexicana plant and optionally containing an aqueous, ethanolic or aqueous-ethanolic extract obtained from the fruits of Cuminum cyminum plant.

The invention further relates to a method of treatment and prevention of the abovementioned disorders, in particular psoriasis comprising administering to a mammal through the oral route or by topical application of the herbal composition containing the fractions obtained from leaves and/or stem of Argemone mexicana plant.

The invention also relates to an aqueous, ethanolic or aqueous-ethanolic extract obtained from the fruits of Cuminum cyminum plant, and provides mechanism of action of the Cuminum cyminum plant extract acting on enzyme inhibition, such as phosphodiesterase (III, IV and V) inhibition and 5-Lipoxygenase inhibition.

### DESCRIPTION OF THE ABBREVATIONS/NOTATIONS

The following abbrevations/notations used throughout the text refer to the following:

[1]: Aqueous (water) extract of the leaves and/or stem of Argemone mexicana plant and the fruits of Cuminum cyminum plant,

[2]: Dry powder obtained on lyophilization of the aqueous (water) extract of the leaves and/or stem of Argemone mexicana plant, and the fruits of Cuminum cyminum plant

[3]: Dry powder obtained on lyophilization of the aqueous (water) extract of the leaves and/or stem of Argemone mexicana plant,

[4]: Dry powder obtained on lyophilization of the Aqueous (water) extract of the fruits of Cuminum cyminum plant,

[5]: n-butanol-soluble fraction of the aqueous (water) extract of the leaves and/or stem of Argemone mexicana plant,

[6]: Methanol-soluble fraction of the aqueous (water) extract of the leaves and/or stem of Argemone mexicana plant, and

[7]: Methanol-insoluble fraction of the aqueous (water) extract of the leaves and/or stem of Argemone mexicana plant.

### BACKGROUND OF THE INVENTION

Skin disorders like psoriasis are characterized by inflammatory and abnormal epidermal keratinocyte hyper-proliferation resulting in hyperplasia, thickening of the epidermis and presence of red scale plaques. The chronic skin condition recognized for its peculiar clinical symptoms, characterized by circumscribed red patches covered with white scales result in itchy flaky skin. Psoriasis is a very visible disease and frequently affects the face, scalp, trunk and limbs. The lesions in this chronic disease typically are subjected to remissions and excerbations. Although, psoriasis manifests as a skin disorder, it is believed to be a disease of impaired or defective cell mediated immunity. Currently, psoriasis is portrayed as an autoimmune disease, where activated T-lymphocytes, producing multiple cytokines cause secondary epithelial abnormalities. Dysregulated lymphocytes produce cytokines that stimulate the proliferation of apoptosis-resistant keratinocytes. Psoriatic skin lesions are characterized by inflammation, with T cells and neutrophils infiltrating both the dermis and epidermis and excessive scaling related to epidermal hyperproliferation and aberrant keratinocyte differentiation (Reich, et. al., 2001). The defect in psoriasis appears to be overly rapid growth of keratinocytes and shedding of scales from the skin surface. Drug therapy is directed at slowing down this process.

The symptoms observed in psoriatic patients include hyperplasia and abnormal cornification of epidermal cells ascribed to the excess turnover of the cells by hyper metabolism, asthenia of inflammatory response in the epidermal layer, vasodilatation and leukocyte migration and infiltration into the epidermal cell layers (Beutner, 1982). However, it is now recognized that epidermal hyperplasia is a reaction to the activation of immune system in focal skin regions, which in turn, is mediated by CD8+ and CD4+ T lymphocytes that accumulate in the diseased skin. Indeed, psoriasis is now recognized as the most prevalent T cell-mediated inflammatory disease of humans. Because the clinical appearance of psoriasis is largely caused by epidermal changes, the disease has traditionally been considered one of excessive keratinocyte proliferation and abnormal differentiation. Within psoriatic lesions, the keratinocyte cell cycle time is reduced approximately 8 fold (36 vs. 311 hours in normal skin) and the number of dividing cell is doubled, resulting in a hyperplastic epidermis. More recently, infiltration of T lymphocytes in skin lesions has been recognized to be an integral feature of psoriasis. Current evidence suggests that epidermal changes in psoriasis are caused by actions of T lymphocytes in skin lesions.

Although, psoriasis could tentatively be classified as an inflammatory disease based on the selective accumulation of T lymphocytes in skin lesions, direct evidence shows that T lymphocytes induce or sustain the disease process. It was found that PUVA therapy depleted lymphocytes in concert with disease improvements. These data were consistent with a role for T cells in pathogenesis. Cyclosporine was found to have dramatic effects on disease activity. Since cyclosporine has a major inhibitory effect on T cell activation, arguments began to be made that psoriasis was fundamentally an inflammatory disease.

T lymphocytes must infiltrate the dermis and then adhere to keratinocytes to produce a psoriatic plaque. Hence molecular regulating T cell adhesion and trafficking become tenable therapeutic targets and its role in pathophysiology is of considerable importance. Intravascular adhesion events can be inhibited by blocking chemokine triggering or blocking integrin binding (LFA-1 to ICAM-1). Integrin blockade or reduction of its surface expression could be an important event for lymphocytes trafficking which help in anti-psoriatic therapy.

The number of different and sometimes toxic treatments employed for amelioration of psoriasis is testimony to the resistant nature of this disease. As the majority (90%) of psoriasis patients have limited forms of the disease, topical treatments that include dithranol, tar preparations, corticosteroids and the recently introduced vitamin D3 analogues (calcipotriol, calcitriol) can be used. A minority (10%) of psoriasis patients has a more serious condition, for which a number of systemic therapeutic modalities are available. Specific systemic therapies include UVB, PUVA, methotrexate, vitamin A derivatives (acitretin) and immuno-suppressants such as Cyclosporin A. The effectiveness of Cyclosporin and FK-506 for treating psoriasis provides support for the T cell hypothesis as the prime cause of the disease.

The topical use of corticosteroids reduces the symptoms of psoriasis. However their administration for long period of time, which is necessary in such treatment causes tachyphylaxis so that either the dose has to be increased or stronger drugs has to be used leading to atrophy and achromasia or loss of pigmentation of peripheral normal skin, when it is topically applied on psoriatic lesion (BNF, 2001).

Use of phototherapy (irradiation with ultraviolet radiation) or photochemotherapy, which consists of external or internal administration of psoralens and application of long wave ultraviolet rays to the affected part, is associated with disadvantages like the possibility of accelerated aging or pigmentation of the skin and of inducing carcinogenesis (BNF, 2001).

External use of coal tar, even though is associated with fewer side effects when compared with steroids, is, however, has drawbacks, which include strong odour, staining of skin etc. Occasionally it may cause stimulant dermatitis.

Methotrexate, even though, is a drug of choice for treating psoriatic conditions, however, need to be closely monitored because it can cause liver damage and/or decrease the production of oxygen carrying red blood cells, infection-fighting white blood cells and clot-enhancing platelets. The long-term use of psoralens and methotrexate significantly increase the risk of squamous cell carcinoma in patients with psoriasis (Stern, 1994).

The retinoids such as etretinate are taken internally for patients suffering from intractable psoriasis, however it is teratogenic and likely to accumulate in the body for a long period of time and hence for a person capable of childbearing it should be avoided (BNF, 2001). Use of macrocyclic immunosuppressive agents as cyclosporine, Tacrolimus and Ascomycin may impair kidney function or cause hypertension. Possible side effects of hydroxyurea include anemia and a decrease in white blood cells and platelets.

Calcipotriol, a synthetic vitamin D3 analogue has become one of the widely prescribed treatment of psoriasis. However, it causes significantly more skin irritation than potent topical corticosteroids. The common adverse effects include lesional or perilesional irritation, facial or scalp irritation, or excerbation of psoriasis (Ashcroft, et al., 2000).

Current biotechnology approaches to psoriasis treatment relate to a direct pharmaceutical-mediated attack, either on cell proliferation or on the immune component of the disease. Immunosuppressive immunobiologicals as Clenoliximab, MEDI-507, ICM3, IDEC-114, SMART Anti-CD3, Zenapax Amavive, Hul 134, Xanelim, HuMaxCD4, IC747, IDEC-114 IDEC-131, Nuvion, DAB3891L-2, ONTAK and Etarnercept, known to block immune responses at various stages are currently under different phases of clinical trials.

Many treatments are available for treatment of psoriasis. Yet none of them are universally safe and effective. The magnitude of the impact of psoriasis is similar to that of other diseases like depression, hypertension and congestive heart failure. The cost of treating the disease averages 800 USD per patients per year in the United States, and the disease can contribute significantly to loss in productivity (Feldman, 2000). Despite the availability of various treatments, psoriasis owing to its sporadic course, variable response to treatments, and adverse effects is a difficult disease to cure. The devastating nature of psoriasis is emphasized by the extent of these side effects that disease sufferers are willing to endure to attain a remission to a disease that they know will recur sooner or later.

Apart from the clinical manifestations and the inconvenience the psychological impact of the disease on the patient's life is tremendous. Psoriasis is a complex condition affecting all aspects of emotion and physical debilitation for the patient, which leads to detraction significantly from the quality of life (Fortune, et al., 1998). Skin disease like psoriasis substantially reduces the quality of life for millions of people all over the world. Moreover, as it is often clearly visible, affected individuals suffer marked distress, embarrassment and discomfort.

A great need, if not imperative exists for a new medication for the treatment of psoriasis, which addresses the shortcomings and limitations of the current therapy and thereby, provides complete cure without any relapse and having no serious adverse reactions and which moreover, is safe, effective, well-tolerated and non-toxic when administered to human beings suffering from psoriasis.

Since times immemorial plants have been a rich source of food and medicine for man. Many parts of plants have been and are being used in a number of ways by man in his regular lifestyle. Plants or parts thereof have been used to prepare teas or other concoctions/preparations for oral consumption. Many times these have been used for local application also.

One example of a plant being used for medicinal purposes is Argemone mexicana. This plant is also known as Mexican Poppy or Prickly Poppy, Bhatkateya, Bharbhand or Satyanashi. This has been used for medicinal purpose in Ayurveda in India. It is reported to be an anodyne, an aperient, a carminative, a cathartic, a demulcent, a depurative, a diuretic, an emetic, an emmenagogue, an expectorant, a hemostat, a laxative, a narcotic, a purgative, a sedative, a stimulant, a sudorific, and a vulnerary (Duke, J. A., 2001). Argemone mexicana is a folk remedy for cancer, catarrh, chancre, cholecystitis, cold, colic, dysuria, eruptions, excrescences, eyes, fever, headache, herpes, inflammation, itch, ophthalmia, parturition, pinkeye, rheumatism, scabies, skin ailments, tooth ache and warts. The plant reportedly destroys worms, cures itching, leprosy, leukoderma, inflammations, bilious fevers, useful in strangury, an antidote to various poisons. It enriches blood, is a good expectorant and an aphrodisiac (Kirtikar, K. R., et. al., 1933) and is useful in colds and pneumonia (Oakes, A. J., et al., 1958). Urinary infections can be treated with a decoction of whole plant of Argemone mexicana along with roots of Latania verschaffeltii plant and leaves and stem of Caesalpinia bonduc plant (Gurib-Fakim, A., et al., 1996) or by using a tea made from boiling Argemone mexicana plant (Elridge, J., 1975). Whole plant including roots is boiled and consumed in thick consistency for curing hepatitis and taken as a tea to cleanse body, improve fluid balance and regulate urination (Halberstein, R. A., et. al., 1978). A drink made by boiling Argemone mexicana plant with chips from Bursera simarub plant is useful for high blood pressure (Asprey, G. F., et. al., 1955). The plant is used as a purge in Costa Rica and as a cure for drunkenness in Guetamala. Curacao natives take young leaf tea for asthma, cardosis, cough and fever. The Bahamians use it for hepatitis; the Jamaicans for cold and fever; the Yucatanese for hepatosis, jaundice and pertussis; while the Venezuelans use it for cancer, epilepsy and malaria. The latex of the plant is widely used for ophthalmia, ringworms, scabies and warts.

Inspite of the fact that Argemone mexicana has been known and has been widely used, little or no documentation exists describing the various medicinal uses of the plant, the illnesses being treated or the effectiveness of the plant.

Even though a variety of medicinal uses have been attributed without acceptable scientific evidence to different parts of Argemone mexicana plant and prior art reports the presence of alkaloids, flavonoids, amino acids, organic acids and sugars in the plant, the active component(s) of Argemone mexicana responsible for the said medicinal activities are not known and clearly defined.

It must be overly emphasized that the prior art does not teach or suggest the use of the leaves and stem of Argemone mexicana plant systematically for treatment of psoriasis and diseases which occur through similar biochemical and immunological disorders. For instance, the use of an extract prepared from the leaves or stem of Argemone mexicana plant for the treatment of psoriasis by oral administration or as a topical application is hitherto not known and therefore, is novel and inventive.

The extracts of the leaves or the stem of the Argemone mexicana plant, optionally containing an extract of Cuminum cyminum plant were selected for proof of concept study. The extracts and fractions of the leaves or the stem of the plant, were taken up for another proof of concept study and mechanistic studies.

### OBJECTS OF THE INVENTION

It is therefore, an object of the present invention to provide a novel composition for treatment of psoriasis in mammals.

Another object of the present invention is to provide a novel composition for treatment of various other disorders, such as skin ailments plaque psoriasis, guttate psoriasis, pustular psoriasis and psoriasis of the nails; dermatitis and scleroderma; eczema; inflammatory disorders and other autoimmune diseases like psoriatic arthritis, rheumatoid arthritis, Crohn's disease, multiple sclerosis, initable bowel disease, ankylosing spondilitis, systemic lupus erythremetosus and Sjogren's syndrome; allergies like asthma and chronic obstructive pulmonary disease.

Another object of present invention is to provide a novel composition for treatment of various disorders, in particular psoriasis in mammals, which is safe, well-tolerated, non-toxic, with minimal and reversible adverse reactions or side effects and most importantly, which minimizes relapse or recurrence of the disease following completion of a treatment regimen.

Yet another object of present invention is to provide a composition for treatment of various disorders, in particular psoriasis in mammals, which is safe, well-tolerated, non-toxic, with minimal and reversible adverse reactions or side effects, and most importantly, which minimizes relapse or recurrence of the disease following completion of a treatment regimen, suitable as an oral administration or as a topical application.

A further object of the present invention is to provide a process for preparation of the composition, which is selective, simple, efficient and cost-effective.

Yet further object of the present invention is to provide a method for treatment of various disorders, in particular psoriasis in mammals comprising administration of a composition which is safe, well-tolerated, non-toxic, with minimal and reversible adverse reactions or side effects, and most importantly, which minimizes relapse or recurrence of the disease following completion of a treatment regimen, suitable as an oral administration or as a topical application.

### SUMMARY OF THE INVENTION

As mentioned hereinearlier, it must be overly emphasized that the prior art does not teach or suggest the use of the leaves and/or stem of Argemone mexicana plant systematically for treatment of various disorders, eg. skin ailments such as psoriasis, dermatitis and scleroderma; inflammatory disorders and other autoimmune diseases like psoriatic arthritis, rheumatoid arthritis, Crohn's disease, multiple sclerosis, irritable bowel disease, ankylosing spondilitis, systemic lupus erythremetosus and Sjogren's syndrome; allergies like asthma and chronic obstructive pulmonary disease.

The present inventors have found that the leaves and/or stem of Argemone mexicana plant can be extracted with water, ethanol or a mixture of water and ethanol and the extracts thus obtained exhibit useful in vitro and in vivo immunological and pharmacological activities, hitherto not known and thereby, provide a novel medication/composition for the treatment and prevention of psoriasis and other disorders, the said medication/composition exhibiting significant reduction in the rate of PASI score with better tolerability within the range of normal permissible limit and which moreover, is safe, well-tolerated, non-toxic, with minimal and reversible adverse reactions or side effects, and most importantly, which minimizes relapse or recurrence of the disease following completion of a treatment regimen.

Again, the present inventors have found that the water, ethanol or a mixture of water and ethanol extracts obtained from the leaves and/or stem of Argemone mexicana plant can be optionally combined with an aqueous, ethanolic or aqueous-ethanolic extract obtained from the fruits of Cuminum cyminum plant, and that the combination also exhibits useful in vitro and in vivo immunological and pharmacological activities, hitherto not known and thereby, provide a novel medication/composition for the treatment and prevention of psoriasis and other disorders, the said medication/composition exhibiting significant reduction in the rate of PASI score with better tolerability within the range of normal permissible limit and which moreover, is safe, well-tolerated, non-toxic, with minimal and reversible adverse reactions or side effects, and most importantly, which minimizes relapse or recurrence of the disease following completion of a treatment regimen.

Additionally, the present inventors have found that the extracts obtained from the leaves and/or stem of Argemone mexicana plant, optionally in combination with an aqueous, ethanolic or aqueous-ethanolic extract obtained from the fruits of Cuminum cyminum plant can be prepared by a selective process, wherein substantially all of the compounds, for instance mixture of alkaloids, flavonoids, organic acids, amino acids, sugars and salts, present in the parts of the plant used are extracted.

Again, the present inventors have found that the extracts obtained from the leaves and/or stem of Argemone mexicana plant thus obtained could be fractionated into several fractions, again by a selective process and the fractions contain substantially all of the compounds, for instance mixture of alkaloids, flavonoids, organic acids, amino acids, sugars and salts, contained in the original extract are retained in the fractions.

Additionally, present inventors have found that the fractions of the extracts obtained from the leaves and/or stem of Argemone mexicana plant exhibit useful in vitro and in vivo immunological and pharmacological activities, hitherto not known and thereby, provide a novel medication/composition for the treatment and prevention of psoriasis and other disorders, the said medication/composition exhibiting significant reduction in the rate of PASI score with better tolerability within the range of normal permissible limit and which moreover, is safe, well-tolerated, non-toxic, with minimal and reversible adverse reactions or side effects, and most importantly, which minimizes relapse or recurrence of the disease following completion of a treatment regimen.

The present inventors, have further found that the extracts obtained from the leaves and/or stem of Argemone mexicana plant, and the fractions obtained from the extracts obtained from the leaves and/or stem of Argemone mexicana plant, exhibit useful in vitro and in vivo which exhibit immunosuppression, lymphoproliferation inhibition, cytokine modulation such as IL-2 inhibition, IFNgamma inhibition, IL-10 induction, keratinocyte proliferation inhibition, keratolytic activity, endothelial cell proliferation inhibition, inhibition of cell adhesion molecule expression such as ICAM-1, MEST inhibition, and enzymes inhibition such as p60src Tyrosine kinase , which are known to be involved in anti-psoriatic activity .

Even though, several solvents including water can be used for fractionation of the extracts obtained from the leaves and/or stem of Argemone mexicana plant the present inventors have found that only solvents like n-butanol and methanol provide three distinct fractions, viz. a n-butanol-soluble fraction, a methanol-soluble fraction and a methanol-insoluble fraction, which in addition to containing the major class of organic compounds including alkaloids, amino acids, sugars and salts exhibit the aforesaid useful in vitro and in vivo immunological and pharmacological activities, which hitherto not known are involved in anti-psoriatic activity.

Thus, the process for preparation of an extract prepared from the leaves and/or of Argemone mexicana plant and the process for selective preparation of fractions of the extracts, which is efficient and economical and the use of the extracts and the fractions for the treatment of various disorders including psoriasis are both hitherto not known and therefore, is novel and forms the inventive step of the present invention.

Further, the present inventors have surprisingly found that the aforesaid extracts and the fractions obtained from the leaves and/or stem of Argemone mexicana plant can be formulated into a novel composition, suitable for oral administration as well as topical application and thereby, providing a novel method for treatment and prevention of psoriasis, and other disorders, which is safe, well-tolerated, non-toxic, with minimal and reversible adverse reactions or side effects, and most importantly, with minimal relapse or recurrence of the disease following completion of a treatment regimen, which forms the basis of another inventive merit of the present invention.

Finally, the present inventors have found to their surprise that the abovementioned herbal composition can optionally contain an extract obtained from the fruits of Cuminum cyminum plant, which does not diminish the usefulness and effectiveness of the herbal composition for treatment and prevention of psoriasis and other disorders such as psoriatic arthritis, rheumatoid arthritis, Crohn's disease, multiple sclerosis, irritable bowel disease, ankylosing spondilitis, systemic lupus erythremetosus, Sjogren's syndrome, allergies like asthma and chronic obstructive pulmonary disease, which forms yet another basis of another inventive merit of the present invention.

Thus, in accordance with the above the present invention provides,

i) a novel herbal composition useful in the treatment and prevention of psoriasis including plaque psoriasis, gutatte psoriasis, pustular psoriasis and psoriasis of the nails; dermatitis and scleroderma; eczema; inflammatory disorders and other autoimmune diseases like psoriatic arthritis, rheumatoid arthritis, Crohn's disease, multiple sclerosis, irritable bowel disease, ankylosing spondilitis, systemic lupus erythremetosus and Sjogren's syndrome; allergies like asthma and chronic obstructive pulmonary disease;

ii) which is safe, well-tolerated, non-toxic, with minimal and reversible adverse reactions or side effects, and most importantly, with minimal relapse or recurrence of the disease following completion of a treatment regimen;

iii) which contains as active ingredient/s an aqueous, ethanolic or aqueous-ethanolic extract obtained from the leaves and/or stem of Argemone mexicana plant, optionally in combination with an aqueous, ethanolic or aqueous-ethanolic extract obtained from the fruits of Cuminum cyminum plant in conjunction with pharmaceutically acceptable carriers , which exhibit useful in vitro and in vivo immunological and pharmacological activities, immunosuppression, lymphoproliferation inhibition, cytokine modulation such as IL-2 inhibition, IFNgamma inhibition, IL-10 induction, keratinocyte proliferation inhibition, keratolytic activity, endothelial cell proliferation inhibition, inhibition of cell adhesion molecule expression such as ICAM-1, MEST inhibition, and enzymes inhibition such as p60src Tyrosine kinase, which are known to be involved in anti-psoriatic activity; or

iv) which contains as active ingredient/s a n-butanol-soluble fraction, a methanol-soluble fraction or a methanol-insoluble fraction obtained from the aqueous, ethanolic or aqueous-ethanolic extract obtained the leaves and/or stem of Argemone mexicana plant, in conjuction with pharmaceutically acceptable carriers, which exhibit useful in vitro and in vivo immunological and pharmacological activities, immunosuppression, lymphoproliferation inhibition, cytokine modulation such as IL-2 inhibition, IFNgamma inhibition, IL-10 induction, keratinocyte proliferation inhibition, keratolytic activity, endothelial cell proliferation inhibition, inhibition of cell adhesion molecule expression such as ICAM-1, MEST inhibition, and enzymes inhibition such as p60src Tyrosine kinase , which are known to be involved in anti-psoriatic activity; or

v) a novel composition containing aqueous, ethanolic or aqueous-ethanolic extract obtained from the leaves and/or stem of Argemone mexicana plant, containing a mixture of alkaloids, flavonoids, organic acids, amino acids, sugars and salts, possessing in vitro human keratinocyte proliferation inhibition, human dermal microvascular endothelial cells (HDMEC) proliferation inhibition and mitogen induced lymphoproliferation inhibition properties and thereby, useful for treatment of psoriasis and related diseases wherever immunosuppression is required. Human dermal microvascular endothelial cells (HDMEC) proliferation inhibition can also be used for the tumor conditions wherever excessive angiogenesis is present;

vi) a novel composition containing a n-butanol-soluble fraction, a methanol-soluble fraction or a methanol-insoluble fraction obtained from the aqueous extract obtained the leaves and/or stem of Argemone mexicana plant, containing a mixture of alkaloids, flavonoids, organic acids, amino acids, sugars and salts, possessing in vitro human keratinocyte proliferation inhibition, human dermal microvascular endothelial cells (HDMEC) proliferation inhibition and mitogen induced lymphoproliferation inhibition properties and thereby, useful for treatment of psoriasis and related diseases wherever immunosuppression is required. Human dermal microvascular endothelial cells (HDMEC) proliferation inhibition can also be used for the tumor conditions wherever excessive angiogenesis is present;

vii) a novel composition containing aqueous, ethanolic or aqueous-ethanolic extract obtained from leaves and/or stem of Argemone mexicana plant, containing a mixture of alkaloids, flavonoids, organic acids, amino acids, sugars and salts, possessing anti-psoriatic activity mediated through (interleukin-2) IL-2, (Interferon gamma) IFN gamma inhibition property for the use in autoimmune diseases like psoriatic arthritis, rheumatoid arthritis, Crohn's disease, multiple sclerosis, iritable bowel disease, ankylosing spondilitis, systemic lupus erythremetosus, Sjogren's syndrome, allergies like asthma, chronic obstructive pulmonary disease and related conditions;

viii) a novel composition containing a n-butanol-soluble fraction, a methanol-soluble fraction or a methanol-insoluble fraction obtained from the aqueous extract obtained the leaves and/or stem of Argemone mexicana plant, containing a mixture of alkaloids, flavonoids, organic acids, amino acids, sugars and salts, possessing anti-psoriatic activity mediated through (interleukin-2) IL-2, (Interferon gamma) IFN gamma inhibition property for the use in autoimmune diseases like psoriatic arthritis, rheumatoid arthritis, Crohn's disease, multiple sclerosis, ankylosing spondilitis, systemic lupus erythremetosus, Sjogren's syndrome, allergies like asthma, chronic obstructive pulmonary disease and related conditions;

ix) a novel composition containing aqueous, ethanolic or aqueous-ethanolic extract obtained from leaves and/or stem of Argemone mexicana plant, containing a mixture of alkaloids, flavanoids, organic acids, amino acids, sugars and salts, possessing Interleukin-10 (IL-10) inducer property, which is useful for treatment and prevention of psoriasis;

x) a novel composition containing a n-butanol-soluble fraction, a methanol-soluble fraction or a methanol-insoluble fraction obtained from the aqueous extract obtained the leaves and/or stem of Argemone mexicana plant, containing a mixture of alkaloids, flavonoids, organic acids, amino acids, sugars and salts, possessing Interleukin-10 (IL-10) inducer property, which is useful for treatment and prevention of psoriasis;

xi) a novel composition containing aqueous, ethanolic or aqueous-ethanolic extract or obtained from leaves and/or stem of Argemone mexicana plant, containing a mixture of alkaloids, flavonoids, organic acids, amino acids, sugars and salts, possessing p70src tyrosine kinase inhibition, which is useful for the treatment and prevention of psoriasis;

xii) a novel composition containing a n-butanol-soluble fraction, a methanol-soluble fraction or a methanol-insoluble fraction obtained from the aqueous extract obtained the leaves and/or stem of Argemone mexicana plant, containing a mixture of alkaloids, flavonoids, organic acids, amino acids, sugars and salts, possessing p70src tyrosine kinase inhibition, which is useful for the treatment and prevention of psoriasis;

xiii) a novel composition containing aqueous, ethanolic or aqueous-ethanolic extract obtained from leaves and/or stem of Argemone mexicana plant, containing a mixture of alkaloids, flavonoids, organic acids, amino acids, sugars and salts, possessing (Inter cellular adhesion molecule-1) ICAM-1 cell adhesion molecule expression inhibition on endothelial cells for the use in inhibiting immune cell including lymphocytes, monocytes and neutrophils trafficking, which is useful for the treatment and prevention of psoriasis;

xiv) a novel composition containing a n-butanol-soluble fraction, a methanol-soluble fraction or a methanol-insoluble fraction obtained from the aqueous extract obtained the leaves and/or stem of Argemone mexicana plant, containing a mixture of alkaloids, flavonoids, organic acids, amino acids, sugars and salts, possessing (Inter cellular adhesion molecule-1) ICAM-1 cell adhesion molecule expression inhibition on endothelial cells for the use in inhibiting immune cell including lymphocytes, monocytes and neutrophils trafficking, which is useful for the treatment and prevention of psoriasis;

xv) a novel composition containing aqueous, ethanolic or aqueous-ethanolic extract obtained from leaves and/or stem of Argemone mexicana plant, containing a mixture of alkaloids, flavonoids, organic acids, amino acids, sugars and salts, possessing in vivo immunosuppression activity in mouse ear swelling test, which is useful for treatment and prevention of psoriasis and other autoimmune disease disorders like psoriatic arthritis, rheumatoid arthritis, Crohn's disease, multiple sclerosis, iritable bowel disease, ankylosing spondilitis, systemic lupus erythremetosus, Sjogren's syndrome, allergies like asthma, chronic obstructive pulmonary disease and related conditions;

xvi) a novel composition containing a n-butanol-soluble fraction, a methanol-soluble fraction or a methanol-insoluble fraction obtained from the aqueous extract obtained the leaves and/or stem of Argemone mexicana plant, containing a mixture of alkaloids, flavonoids, organic acids, amino acids, sugars and salts, possessing in vivo immunosuppression activity in mouse ear swelling test, which is useful for treatment and prevention of psoriasis and other autoimmune disease disorders like psoriatic arthritis, rheumatoid arthritis, Crohn's disease, multiple sclerosis, ankylosing spondilitis, systemic lupus erythremetosus, Sjogren's syndrome, allergies like asthma, chronic obstructive pulmonary disease and related conditions;

xvii) a process for selective preparation of the aqueous, ethanolic or aqueous-ethanolic extract from the leaves and/or stem of Argemone mexicana plant, comprising the steps of extraction of the leaves and stem of Argemone mexicana plant in the form of a coarse paste, with water, ethanol or mixtures thereof respectively, and drying of the extracts to give a powder;

xviii) a process for selective preparation of the aqueous, ethanolic or aqueous-ethanolic extract from the leaves and/or stem of Argemone mexicana plant, optionally in combination with an aqueous extract obtained from the fruits of Cuminum cyminum plant, comprising the steps of extraction of the leaves and stem of Argemone mexicana plant and the fruits of Cuminum cyminum plant, both in the form of a coarse paste, with water, ethanol or mixtures thereof respectively;

xix) a process for selective preparation of a n-butanol-soluble fraction of the leaves and/or stem of Argemone mexicana plant containing a mixture of alkaloids, flavonoids and other organic compounds, comprising fractionation of the aqueous extract of the leaves and/or stem of Argemone mexicana plant between n-butanol and water;

xx) a process for selective preparation of a methanol-soluble fraction of the leaves and/or stem of Argemone mexicana plant containing a mixture of amino acids, organic acids and salts, comprising addition of methanol to the aqueous layer obtained on fractionation of the of the leaves and stem of Argemone mexicana plant with n-butanol and water and separation of the precipitated solids. The supernatent solution constitutes the methanol-soluble fraction;

xxi) a process for selective preparation of a methanol-insoluble fraction of the leaves and/or stem of Argemone mexicana plant containing a mixture of organic acids, sugars and salts, comprising addition of methanol to the aqueous layer obtained on fractionation of the of the leaves and/or stem of Argemone mexicana plant with n-butanol and water and separation of the precipitated solids. The precipitated solid, dried and redissolved in water constitutes the methanol-insoluble fraction;

xxiii) a process for selective preparation of the aqueous, ethanolic or aqueous-ethanolic extract from the leaves and/or stem of Argemone mexicana plant in the form of a coarse paste, which is simple, efficient and cost-effective;

xxiv) a process for selective preparation of a n-butanol-soluble fraction of the leaves and/or stem of Argemone mexicana plant containing a mixture of alkaloids, flavonoids and other organic compounds, comprising fractionation of the aqueous extract of the leaves and/or stem of Argemone mexicana plant between n-butanol and water; which is simple, efficient and cost-effective;

xxv) a process for selective preparation of a methanol-soluble fraction of the leaves and/or stem of Argemone mexicana plant containing a mixture of amino acids, organic acids and salts, comprising addition of methanol to the aqueous layer obtained on fractionation of the of the leaves and/or stem of Argemone mexicana plant with n-butanol and water and separation of the precipitated solids. The supernatent solution constitutes the methanol-soluble fraction, which is simple, efficient and cost-effective;

xxvi) a process for selective preparation of a methanol-insoluble fraction of the leaves an/or stem of Argemone mexicana plant containing a mixture of organic acids, sugars and salts, comprising addition of methanol to the aqueous layer obtained on fractionation of the of the leaves and/or stem of Argemone mexicana plant with n-butanol and water and separation of the precipitated solids. The precipitated solid, dried and redissolved in water constitutes the methanol-insoluble fraction, which is simple, efficient and cost-effective;

xxvii) an aqueous, ethanolic or aqueous-ethanolic extract obtained from the fruits of Cuminum cyminum plant acting on enzyme inhibition, such as phosphodiesterase (III, IV and V) inhibition and 5-Lipoxygenase inhibition.

xxviii) a process for preparation of the aqueous, ethanolic or aqueous-ethanolic extract obtained from the fruits of Cuminum cyminum plant comprising the steps of extraction of the fruits of Cuminum cyminum plant with water, ethanol or mixtures thereof respectively, and drying of the extracts to give a powder;

xxix) a process for preparation of the novel composition containing aqueous, ethanolic or aqueous-ethanolic extract of the leaves and/or stem of Argemone mexicana plant, containing a mixture of alkaloids, flavonoids, organic acids, amino acids, sugars and salts, in conjuction with pharmaceutically acceptable excipients;

xxx) a process for preparation of the novel composition containing aqueous, ethanolic or aqueous-ethanolic extract of the leaves and/or stem of Argemone mexicana plant, containing a mixture of alkaloids, flavonoids, organic acids, amino acids, sugars and salts, optionally in combination with an aqueous extract obtained from the fruits of Cuminum cyminum plant, in conjuction with pharmaceutically acceptable carriers;

xxxi) a process for preparation of the novel composition containing a n-butanol soluble fraction, a methanol-soluble fraction or a methanol-insoluble fraction obtained from the aqueous extract of the leaves and/or stem of Argemone mexicana plant in conjuction with pharmaceutically acceptable carriers;

xxxii) a method for treatment of psoriasis and other disorders, like psoriatic arthritis, rheumatoid arthritis, Crohn's disease, multiple sclerosis, ankylosing spondilitis, systemic lupus erythremetosus, Sjogren's syndrome, allergies like asthma and chronic obstructive pulmonary disease comprising administering orally or as a topical application, to a patient a therapeutically effective amount of the novel composition of this invention.

### DETAILED DESCRIPTION OF THE INVENTION

The invention is detailed as hereinbelow

1) The Plant: Argemone mexicana

The plant used in the present invention is Argemone mexicana. The preferred plant is Argemone mexicana Linn., hereinafter referred to as Argemone mexicana. The herb is a very common weed in the agricultural and wastelands naturalised throughout India up to a height of 1500 m. The mature plant is an erect, prickly annual herb, up to 1.2 m in height. Leaves are sessile, semi-amplexicule, sinuately pinatifid, spiny on margins, mid-rib and veins beneath, flowers are yellow, 2.5-7.5 cms in diameter, capsules are elliptic or oblong-prickly, rarely unarmed, seeds are small, round and blackish brown, deeply reticulate-scrobiculate.

Argemone mexicana is also known by a number of common or vernacular names including Mexican Poppy, Prickly Poppy, Bharbhand, Satyanashi, Shialkanta and Bhatkateya.

The various compounds present in Argemone mexicana plant include inter alia,

i) Alkaloids such as protopine, protopine nitrate, berberine, berberine nitrate, cryptopine, allocryptopine, coptisine, sanguinarine, dihydrosanguinarine, norsanguinarine, 6-acetonyl dihydrosanguinarine, dihydrochelerythrine, chelerythrine, norchelerythrine, 6-acetonyl dihydrochelerythrine, (−) cheilanthifoline, (−)-β-scoulerine methohydroxide, (−)-α-stylopine methohydroxide, 6-acetonyl dihydrosanguinarine, (−)-α-tetrahydropalmatine methohydroxide, reticuline, thalifoline, muramine, argemonine, norargeminine, helleritrine, and oxyhydrastinine;

ii) Flavonoids, such as isorhmanetin, isorhamnetin-3-glucoside and isorhamnetin-3,7-diglucoside;

iii) Fatty acids, such as palmitic, stearic, arachidic, oleic, linoleic, lauric, behenic, lignoceric, hexadecenoic and ricinoleic acids, 11-oxo-triacontanoic acid and 11-hydroxy triacontanoic acid;

iv) Amino acids, such as histidine, lysine, glutamic acid, glycine, alanine, leucine, valine, phenyl alanine, tyrosine, threonine, arginine, serine, asparagine, cysteine, methionine, tryptophan, hydroxyproline, proline and aspartic acid;

v) Carbohydrates, such as glucose and fructose and glycosides.

vi) Organic acids, such as succinic, citric, tartaric and malic acids; and

vii) Other compounds like ceryl alcohol, β-sitosterol, potassium nitrate, calcium phosphate and calcium sulphate.

2) Preparation of the Extracts of the leaves and Stem of Argemone Mexicana Plant

The extract of present invention is harvested from the leaves and/or stem of Argemone mexicana plant. The leaves are preferably collected fresh. The leaves and the stem are cleaned with water, pulverized and blend into a paste.

The extract of the invention can be prepared by a number of extraction procedures. Suitable procedures include, for example maceration, percolation, and extraction using supercritical fluids, liquefied gases or suitable solvents. Typically, the extraction procedure provides an extract containing substantially all the water-soluble compounds present in the plant.

Suitable solvents for extraction of the leaves and stem of the plant are water and alcohols, specially ethanol or mixtures of water and the alcohol.

A preferred extraction procedure is a multi-step, successive maceration and percolation process using water, ethanol and mixtures thereof. The extraction is preferably carried out at room temperature. The leaves and stem of the plant are ground and percolated with water, ethanol or a mixture of water and ethanol. The ratio of plant to the solvent of extraction is 0.5 ml-3 ml per gram of the plant material, preferably about 1 ml per gram of the plant material.

The aqueous, ethanolic or aqueous-ethanolic extract containing the soluble constituents of the plant are combined to produce a single extract solution. The solvent can be removed by conventional procedures at low temperatures preferably below 50° C. under reduced pressure to reduce the volume and increase the concentration of the extracted compounds. The evaporation or concentration process thus used is preferably selected and carried out under conditions to minimize loss of the volatile compounds. To ensure stability of compounds efficient cooling is essential.

The resulting aqueous extract [3] from the leaves and/or stem Argemone mexicana plant contains a total base number between 275 to 345.

A preferred percolation extraction process uses successive stage extraction with aqueous, ethanolic or aqueous-ethanolic phase. The leaves and stem of the plant to be processed are generally pound into a paste. The extraction is carried out by placing the ground plant material into a vessel and mixing with sufficient amount of water, ethanol or mixtures thereof to cover the plant material. The mixture of the plant material and water, ethanol or mixtures thereof is allowed to stand at room temperature to absorb the menstruum and dissolve the entire water-soluble material. The plant material is taken in a percolator with a provision that the plant extract can pass through the bottom.

The percolator is then covered and the plant material allowed to macerate for sufficient time to dissolve the soluble components. Generally the plant material is allowed to percolate for about 12-24 hrs at room temperature and preferably for about 16 hrs. The solvent is then taken out in a collection vessel, while adding another sufficient amount of the same solvent to the percolator to extract substantially all soluble compounds,

Even though, water, ethanol and mixtures thereof extract substantially all soluble compounds present in the plant, for cost purposes and ease of operation water is the most preferred solvent for extraction.

Generally, the amount of water used for extraction of the leaves and stem of the plant is about 0.5 to 3 ml per gram of the leaves and stem of the plant, preferably 1 ml per gram of the leaves and stem of the plant material is used. The yield of the extract by this method is about 5 to 8% on dry weight basis. The extract is preferably filtered and centrifuged.

The resulting extract from this process comprises of the water-soluble components of the plant including alkaloids, flavonoids, amino acids, organic acids, sugars and salts as mentioned hereinearlier.

The extraction process is preferably carried out using a sufficient volume of solvent and over a period of time to extract substantially all the water-soluble compounds in the plant material so that the final extract contains substantially all of the water-soluble compounds.

In a preferred embodiment, the extract obtained is reduced to one fifth of its original volume under reduced pressure at a temperature below 50° C. Typically, it can be further dried by lyophilization or spray drying or in a rotary evaporator to give a greenish-brown powder. Lyophilization is preferred.

This aqueous extract [3] contains alkaloids, flavonoids, organic acid, amino acids, sugars and salts and major class of chemical component present in the plant.

Optionally, the leaves and/or stem of Argemone mexicana plant can be blended with the fruits of Cuminum cyminum plant, which is ground into a paste prior to extraction with water, ethanol or mixtures thereof.

Alternatively, the fruits of Cuminum cyminum plant ground to a paste can be extracted separately with water, ethanol or mixtures thereof to provide an extract containing substantially all the compounds present in the plant. The extraction procedure comprises a multi-step, successive macreation and percolation process using water, ethanol or mixtures thereof. The extracion is preferably carried out at room temperature.

The ratio of the plant to the solvent of extraction is 1 ml-5 ml per gram of the plant, preferably about 3 ml per gram of the plant.

The extract thus obtained from the fruits of Cuminum cyminum plant can then be mixed in suitable proportions with the extracts obtained from the leaves and stem of Argemone mexicana plant.

3) Preparation of the Fractions of the Extracts of the Leaves/Stem of Argemone Mexicana Plant

The fractionation procedure of the aqueous extracts of the leaves and/or stem of Argemone mexicana plant, is achieved through a multi-step, liquid-liquid partition chromatography, precipitation and drying of extracts and provides fractions having substantially different class of compounds as major components.

The fractionation is preferably carried out at room temperature. The aqueous extract of the leaves and/or stem of Argemone mexicana plant is dissolved in water (0.066 g/ml). To the solution is added n-butanol in 3 portions (3×10 ml per gram), with the layer of n-butanol being separated each time. All the three separated n-butanol layers are mixed together, washed with water and the solvent evaporated under reduced pressure below 50° C. to give a viscous mass of the n-butanol-soluble fraction.

The aqueous wash(es) from the above step is poured into approximately 6-7 times its volume of methanol with agitation. After precipitation of solids, the solution was centrifuged and supernatant was decanted. This methanol-soluble fraction was concentrated under vacuum below 50° C. and lyophilized to give solid methanol-soluble fraction.

The methanol-insoluble precipitate obtained in the above step is dried under vacuum at 40° C. and lyophilized. The solid is further dissolved in water (0.1 g/ml), sonicated, centrifuged and filtered. The filtrate is lyophilized to give water-soluble fraction.

Typically, the plant yields about 3-4.5% of n-butanol-soluble fraction; 46-54% of methanol-soluble fraction and 24-30% of methanol-insoluble or water-soluble fraction.

The resulting fractions prepared from the extract of Argemone mexicana plant contains a total base number between

i) Methanol-soluble fraction [6]:290-340

ii) Methanol-insoluble or water-soluble fraction [7]:350-380

The n-butanol-soluble fraction [5] is found to contain contains alkaloids, flavonoids and other low molecular weight compounds; the methanol-soluble fraction [6] is found to contain amino acids, organic acid and salts; while the methanol-insoluble fraction [7] is found to contain sugars, organic acids and salts.

4) The immunological and Pharmacological properties of the extracts and Fractions of the Leaves and/or Stem of Argemone mexicana Plant.

The aforementioned extracts and fractions, obtained from the leaves and/or stem of Argemone mexicana plant exhibit mitogen induced lymphoproliferation inhibition, human keratinocyte proliferation inhibition, HDMEC proliferation inhibition, cytokine inhibition like IL-2, IFN gamma, cytokine induction like IL-10, p70src Tyrosine Kinase enzyme inhibition, ICAM-1 expression inhibition on endothelial cells and immunosuppression in mouse ear swelling test. As mentioned earlier, the immunomodulatory property of aforesaid extracts and fractions are new and hitherto not known, which constitutes an important aspect of this invention. The extracts and fractions are useful in treatment of certain indications, such as psoriasis, rheumatoid arthritis, multiple sclerosis, irritable bowel syndrome, scleroderma, asthma, chronic obstructive pulmonary disease, atopic dermatitis and allergies. The activities exhibited are useful in immunosuppression. The immunosuppressive activity is believed to be involved in the treatment of psoriasis.

In vitro IL-2, IFNgamma inhibition, IL-10 induction, human keratinocyte proliferation inhibition, HDMEC proliferation inhibition, lymphoproliferation inhibition, ICAM-1 expression inhibition on endothelial cells and tyrosine kinase inhibition reveal that the extracts and fractions of the leaves and stem of Argemone mexicana exhibits immunosuppressive and anti-proliferative properties which have a major mediatory role in its anti-psoriatic activity.

It is now generally accepted that T lymphocytes play an important role in the maintenance of psoriatic plaques. The role of T cells in the pathogenesis of psoriasis was discovered through serendipity, the use of targeted immunotherapies as pathogenic probe, and, more recently, the use of SCID mouse model. Cytokines are highly potent biologically active proteins that play an essential role in intercellular communication. It is likely that some of the new approaches currently under investigation will actually lead to both the registration of new drugs for dermatological treatment, and to supplementation of existing therapeutic options. The expression of several cytokines is upregulated in inflammatory skin diseases, possible mediating inflammatory immune responses and leukocyte chemotaxis. Tumor necrosis factor alpha, IL-8 and GMCSF produced by keratinocytes, as well as eicosanoids, are responsible for leukocyte infiltrates in mature lesion psoriasis (Bos, J. D., 1997). The expression of numerous pro-inflammatory genes is elevated in psoriatic tissue compared with non-lesional skin. Proinflammatory cytokines produced by type 1 lesional T helper (Th1) cells are implicated in the pathogenesis of psoriasis (Austin, L. M., et al., 1999). The response of psoriasis to IL-10 is associated with suppression of cutaneous Th1-cell mediated inflammation, and less expression of IFN gamma and TNF alpha. Remarkably, there was evidence that genetic factors are involved in the clinical response to IL-10. IL-10 is known to be less expressed or absent in psoriasis. This relative IL-10 deficiency may be of major importance for the pathogenesis of psoriasis and that its normalization would be beneficial. Several therapies for psoriasis e.g. UV radiation, monomethylfumerate, led to an increase of IL-10 production by several cell types. Another class of drugs like cAMP elevating agents e.g. iloprost, pentoxifylline, led to an upregulation of IL-10 (Platzer, C., et al., 1995). These agents are known effective in the treatment of psoriasis. Inhibitors of the beta-adrenergic receptor e.g. propanolol or the cycloxygenase e.g. indomethacin often lead to excerbation of psoriasis (Khusru, A., et al., 1998).

IL-10 has great impact on immuno-regulation of Th1 response. It promotes the development of type 2-cytokine patterns by inhibiting the IFN gamma production of T lymphocytes and natural killer cells particularly via the suppression of IL-12 synthesis in accessory cells. Moreover, it suppresses proinflammatory cytokine production and antigen-presenting capacity. Intravenous injection of IL-10 was to inhibit proinflammatory cytokine production (Khusru, A., et al., 1998). Since IL-10 dysregulation may play a key role in psoriasis, the therapeutic effect of IL-10 would be valuable for antipsoriasis therapeutic regimen. Subcutaneous injection of IL-10 directly under the psoriatic plaque led to complete clearance of the lesion in psoriatic patients (Khusru, A., et al., 1998). It has been shown that IL-10 inhibits keratinocytic pro-inflammatory cytokine synthesis. Taking consideration of immunological activity and therapeutic potency of IL-10 in psoriasis, a disease where relative IL-10 deficiency, seems to be of crucial importance. Administration as well as induction of IL-10 may be useful novel approaches for psoriasis treatment.

TH1 response has been proven to be involved in pathophysiology of psoriasis. TH1 cytokine such as IL-2 and IFN gamma are upregulated in psoriasis (Nickoloff, B. J., 1991; Uyemura, K., et al., 1993). These cytokine play a key role in the inflammatory and proliferative processes of psoriasis. Immunosuppressive agents of the macrocyclic class, block IL-2 production and thereby inhibit T cell growth. Inhibition of calcineurin dependent and NFAT mediated suppression of IL-2 production and T cell activation has been proven a successful in psoriasis treatment.

Reduction of activated lymphocytes numbers at site is one of the approaches by several means like IL-2 inhibition, purine and pyrimidine synthesis inhibition of lymphocytes, inhibition of signal transduction pathway after growth factor signal to receptors. The fact that cyclosporine and methotrexate improved psoriatic conditions dramatically (Alice B. Gottlieb, 2001). These drugs affect on proliferation of lymphocyte at lesion. The inhibition of T lymphocytes proliferation is prime focus of immunosuppressive drugs in psoriasis (Baumann, G.; 1992, Mollison, K. W., 1998; Finzi A. F., 1989; Ho, L. J., et al., 1999). Drug acting on IL-2 inhibition like cyclosporine and FK506 (Dupont, E., et al., 1986; Gelfand, E. W., et al., 1987; Bloemena, E., et al., 1988; Henderson, D. J., 1991), IL-2 dependent signal transduction pathway inhibitor like Rapamycin (Thomson, A. W, 1991, Flanagan, W. M., 1993; Duncan, J. I., 1994; Siekierka, J. J., 1994) and nucleotide synthesis inhibitor like Methotrexate are known to inhibit mitogen induced lymphoproliferation inhibition (Boffa, M. J., 1999; Levantine, A., 1975; Bialasiewicz, A. A., 1981).

It has been recognized that chronic inflammation leads to hyper-proliferation of keratinocyte and the agent, which can control inflammation also, controls hyper-proliferation of keratinocytes (Meisner Lorraine, F., 2001; Mollison, K. W., 1998). This is the effect of the compound on immune cells, which indirectly inhibit keratinocytes by inhibiting growth factors required for keratinocytes and derived from lymphocytes. Several Vitamin D3 analogs have been targeted for keratinocyte proliferation. It has been discovered that 1,25(OH) 2D3 has the ability to regulate growth and differentiation in many cell types, including cancer cells, epidermal keratinocytes, and activated lymphocytes (Kragballe, K., 1992). This has set the stage for the development of a new class of compounds with potential usefulness in hyperproliferative and immune-mediated diseases. In addition to 1,25(OH) 2D3, the synthetic vitamin D3 analogues 1 alpha-OH-D3, 1,24(OH) 2D3, and calcipotriol have undergone clinical evaluation. Calcipotriol has been studied most extensively. Their mechanism of action is not yet fully understood and may prove complex. The most important effect may be a direct regulation of keratinocyte proliferation and differentiation. The aim in the study is to see the direct effect of extracts on keratinocyte proliferation.

Expansion of the dermal microvasculature is a prominent feature of psoriasis. Although the pathogenetic process resulting in vascular morphological changes remains unclear, considerable evidence suggests the involvement of angiogenesis in psoriasis (Creamer, D., et al., 1997). Upregulation of NGF in the keratinocytes is also observed in conditions characterized by hyperplasia of keratinocytes lead to scaling and endothelial cells lead to angiogenesis. The role of NGF in inflammation by studying the effects of NGF on endothelial cell proliferation and intracellular adhesion molecule expression by endothelial cells is another niche for psoriasis pathophysiology. Several in vivo and in vitro studies have observed that NGF can induce an inflammatory response. NGF, which are key contributing components of an inflammatory response cause upregulation of ICAM-1 and proliferation of the endothelial cells and keratinocytes. Vascular endothelial growth factor (VEGF) is also a potent mitogen with a unique specificity for endothelial cells and a key mediator of aberrant endothelial cell proliferation (Siemeister, G., et al., 1998). Epidermal proliferation is closely associated with excessive microvascular expansion within the papillary dermis (Kuroda, K., et al., 2001). Inhibition of endothelial cell proliferation will inhibit angiogenesis, which in turn help in suppressing neovascularization and trafficking of immune cells in dermis.

Once T cells become activated in skin, they develop surface proteins, such as common leukocyte antigen (CLA), which allow them to home to skin. Leukocyte trafficking in normal and diseased condition is generated by expression of adhesion molecules, chemokines and other chemotactic compounds. Endothelial cells have important roles in this process. Binding of leukocytes to endothelial cells is the first essential step. Microvascular endothelial cells of human skin contribute to the recruitment of inflammatory leukocytes by expressing inducible leukocyte adhesion molecules such as endothelial leukocyte adhesion molecule-1 (ELAM-1 or E selectin), vascular cell adhesion molecule-1 (VCAM-1), and ICAM-1. Increased expression of ICAM-1 is closely associated with T cell migration in vivo but also contributes to adhesion of granulocytes (Munro, J. M., et al.,1989; Munro, J. M., et. al.,1991; Oppenheimer-Marks, N., et al., 1991). Leukocytes bind to ICAM-1 on endothelial cell surface by its ligand leukocyte function-associated-1 molecule (LFA-1 or CD11a/CD18). Rolling, or slowing down of T lymphocytes within blood vessels, is mediated in part by interactions between selectins and their ligands. Tight adhesion of T lymphocytes to the luminal side of the endothelial cells is mediated in part by interactions of LFA-1 on T cells with ICAM-1 on endothelial cells. Subsequently, to rolling and tight adhesion, the T lymphocytes slip in between neighboring endothelial cells into the dermis (Wakita. H., 1994). Activated T lymphocytes in the dermis, may be of importance in lymphocyte trafficking in the epidermis by the induction of keratinocyte ICAM-1 expression. Expression of ICAM-1 is restricted on resting cells but is highly inducible by activators such as exposure to IL-1 β or IFN-α (Dustin, M. L., et al., 1988; Griffiths, C. E., et al., 1989).

More is now known about trafficking of inflammatory cells in the skin, with specific molecular details involving various cytokines, chemotactic factors, and adhesion molecules. One key element is the in vivo movement of T cells that express LFA-1 into the epidermis, and their subsequent binding to keratinocytes via the surface expression of intercellular adhesion molecule-1 (ICAM-1). This interaction represents a common immunological pathway, which has been identified in a wide variety of different skin diseases. The identification of keratinocyte-derived molecules such as ICAM-1, which influence the chemotaxis and adherence of T cells, adds substantial evidence supporting an active participatory role for keratinocytes in cutaneous immunohomeostasis. Because inflammatory skin diseases are associated with an upregulation of endothelial cell adhesion molecules and because the presence of inflammatory cells in dermis and epidermis is considered an important feature in psoriasis. There is one of the focus to downregulate the expression of ICAM-1 for reducing lymphocyte trafficking. Anti-sense oligodeoxynucleotide for ICAM-1 may be of considerable value in the treatment of psoriasis and other inflammatory skin disorders (Mehta, R. C., et al., 2000).

Lymphocytes bind to keratinocytes after activation with interferon gamma (IFNgamma) and tumor necrosis factor (TNF). Because intercellular adhesion molecule-1 (ICAM-1) is a ligand for LFA-1, we studied the cellular expression of ICAM-1 in cultured endothelial cells. Alicaforsen (ISIS-2302) is an RNase H-dependent antisense inhibitor of the intercellular adhesion molecule ICAM-1 under development by Isis Pharmaceuticals, for the potential treatment of a variety of inflammatory disorders.

The balance of signals that regulate the homeostasis of normal epidermis is altered in psoriasis (Inohara, S., 1992; McKay, I. A., et al., 1995; van Ruissen, F., et al., 1996). In normal epidermis, the basal layer contains progenitor cells responsible for continued local renewal, and although occasional suprabasal mitoses may be observed, it is accepted that the suprabasal cells are committed to terminal differentiation. In the psoriatic plaque there are numerous dividing cells and mitotic figures in several cell layers. It is suggested that epidermal hyperproliferation in psoriasis results either from an increase in cycling cells derived from keratinocyte stem cells, or from an increase in the transient amplifying cell population (van Ruissen, F., et al., 1996; Bata-Csorgo, Z., et al., 1993). Identification of specific signal transduction pathways that mediate proliferative, metabolic and inflammatory processes, such as inhibitors of protein tyrosine kinases (PTKs), a molecules with a central role in the pathogenesis of psoriasis, may play crucial role in anti-psoriatic drug development. PTKs are closely associated with cell growth, proliferation, differentiation and signaling of the immune systems (Hunter, T., et al., 1985; Ullrich, A., et al., 1990). Uncontrolled signaling from receptor tyrosine kinases and intracellular tyrosine kinases can lead to numerous diseases, whereas on the other hand decreased signaling can also lead to disease. Over-signaling of PTKs has been observed in psoriasis. Blocking of PTKs seems to be pivotal in anti-psoriatic treatment. Selective PTK inhibitors to EGFR kinase activity have been reported for effective suppressors of psoriatic keratinocyte growth (Hannah Ben-Bassat, 2001).

From in vivo studies it is observed that the extract prevented the DNFB induced ear swelling, which moreover, is dose dependent, in a mouse ear swelling test model. This clearly indicates immunosuppression.

5) Toxicological Studies

Acute toxicity (LD₅₀) of extract and fractions [3, 5 and 6] was evaluated in mice and rat by oral and i.v. routes. Group of ten (10) animals from each species per route per dose were medicated and results were calculated on day 15.

a) The following Values were Observed for the Aqueous Extract [3]

| | |
|---|---|
| LD₀ of mice p.o. | >5000 mg/kg bwt |
| LD₅₀ of mice i.v. | >1000 mg/kg bwt (50% mortality) |
| LD₀ of rat p.o. | >5000 mg/kg bwt |
| LD₅₀ of rat i.v. | >1000 mg/kg bwt (50% mortality) |

Symptoms Observed in Rats and Mice by i.v. Route:

Dose dependent lethargy, polypnea, convulscence, and prostration were noticed when administered by i.v. route. The animals were normal next day. Sluffing of tail was observed in surviving animals.

No symptoms were observed in mice and rats medicated p.o.

b) For the Methanol-Soluble Fraction [5], the Following Values Were Observed

LD₀ of mice p.o.: >5000 mg/kg bwt

LD₅₀ of mice i.v.: >5000 mg/kg bwt

No symptoms were observed in mice medicated p.o.

c) For the Methanol-Insoluble Fraction [6], the Following Values were Observed

LD₀ of mice p.o.: >5000 mg/kg bwt

LD₅₀ of mice i.v.: >5000 mg/kg bwt

No symptoms were observed in mice medicated p.o.

6) The Novel herbal Composition Containing the Extracts and Fractions of the Leaves and Stem of Argemone mexicana Plant.

The novel composition of the present invention can be prepared suitable for oral administration or suitable for topical application.

Suitable forms of oral administration are those such as tablets, capsules, syrups, elixirs or suspensions.

Suitable forms of topical application are those such as ointments, creams, lotions, oils or transdermal drug delivery systems.

The oral and topical compositions thus prepared comprising the extracts of the leaves and/or stem of the Argemone mexicana plant, either alone or optionally in combination with the extracts of the fruits of Cuminum cyminum plant additionally can be formulated with pharmaceutically acceptable carriers.

Similarly, the oral and topical compositions thus prepared comprising the fractions obtained from the extracts of the leaves and/or stem of the Argemone mexicana plant additionally can be formulated with pharmaceutically acceptable carriers.

The novel composition containing the extracts and fractions may suitably be provided in the form of a liquid, a dry powder or powdered herbal concentrate, capsule, tablet and the like to a mammalian patient for oral administration.

The total amount of the composition prepared and the ratios of the ingredients in the mixture are somewhat variable. A typical mixture as a unit dose for oral administration would consist of approximately 10-12 leaves of Argemone mexicana plant of about 8-10 inch in size and optionally about 10 gm of Cuminum cyminum plant in approximately 50 ml of water, containing about 2 to about 100 mg/gm or ml of the composition containing the fresh extract.

The concentration and amount of the ingredients of the composition typically are as follows:

Composition Containing the Extracts of Argemone mexicana and Cuminum cymimum

In a typical composition, the concentration of the ingredients per gm or ml of the composition are:

| | |
|---|---|
| Extract from the leaves and/or stem of Argemone mexicana plant | 10-50% |
| Extract from fruits of Cuminum cyminum plant | 60-90% |
| or 2 mg to 100 mg per gm or ml of the composition. | |

Composition Containing the Extracts of Argemone Mexicana

The composition may contain the extract of the leaves and/or stem of the Argemone mexicana [3] plant in an amount in the range from between 50 mg and 5000 mg, preferably 1000 mg dose per day.

Composition Containing the Fractions of Argemone Mexicana

The compositions may contain the n-butanol-soluble fraction [5] in an amount in the range from between 5 mg to 200 mg, preferably 40 mg dose per day; the methanol-soluble fraction [6] in an amount in the range from between 25 mg to 2550 mg, preferably 550 mg dose per day; the methanol-insoluble fraction [7] in an amount in the range from between 5 mg to 1250 mg, preferably 280 mg dose per day.

Suitable pharmaceutically acceptable carriers include: Sugars, such as lactose, sucrose, mannitol, sorbitol and xylitol; starches such as corn starch, tapioca starch and potato starch; cellulose and its derivatives such as sodium carboxymethyl cellulose, ethyl cellulose and methyl cellulose; calcium phosphates such as dicalcium phosphate and tricalcium phosphate; sodium sulphate; calcium sulphate; polyvinylpyrrolidone; polyvinyl alcohol; stearic acid; vegetable oils such as peanut oil, cottonsead oil, sesame oil, olive oil and corn oil; non-ionic, cationic and anionic surfactants; ethylene glycol polymers; beta-cyclodextrin; fatty alcohol; hydrolysed cereal solids; as well as other non-toxic compatible fillers, binders, disintegrants, buffers, preservatives, antioxidants, lubricants, flavouring agents and the like commonly used in pharmaceutical formulations.

The novel composition is found to exhibit excellent results in treating psoriasis.

For example, the novel composition may be provided in a formulation containing extract from the leaves and/or stem of Argemone mexicana plant and containing the extracts of the fruits of Cuminum cyminum plant at a concentration between about 2 and 100 mg/gm or ml and it can be administered to a patient for example, in 1-3 gm dosages, once daily for 7 days a week for a maximum period of about 8 to about 20 weeks. In a preferred embodiment 1-3 gm of extract is administered consecutively for three days a week for a period of 12-15 weeks.

A preferred composition for oral administration would comprise the following:

i) 265 mg of aqueous extract of the leaves and/or stem of Argemone mexicana plant+282.25 mg of lactose and 275 mg of colloidal Silicon dioxide.

ii) 550 mg of methanol-soluble fraction of aqueous extract of the leaves and/or stem of Argemone mexicana plant+146.85 mg of lactose and 3.15 mg of colloidal Silicon dioxide.

iii) 280 mg of methanol-insoluble fraction of aqueous extract of the leaves and/or stem of Argemone mexicana plant+366.75 mg of lactose and 3.25 mg of colloidal Silicon dioxide.

iv) 40 mg of n-butanol-soluble fraction of aqueous extract of the leaves and/or stem of Argemone mexicana plant+646.55 mg of lactose and 3.45 mg of colloidal Silicon dioxide.

The above compositions can be blended until uniform and then filled in hard gelatin capsules of the size “00”. The processing area is ideally maintained at 40±5% RH at 18-22° C.

A typical process for preparation of the composition of the invention is illustrated below:

The active ingredient(s), lactose and colloidal silicon dioxide are blended until uniform. They are filled in hard gelatin capsules of size “00”. The processing area is ideally maintained at 40±5% RH at 18-22° C.

When a topical application is administered, the amount of the extract of Argemone mexicana plant ranges from 0.5% to 10% by weight of the extract. The preferred composition contains 4% to 6%. 3

A typical composition for topical application would constitute:

| | |
|---|---|
| Aqueous extract of Argemone mexicana leaves or stem | 5 gm |
| Water | 100 ml |
| Hydroxy propyl methyl cellulose (HPMC) | 4 gm |

The above ingredients were blended slowly to obtain a smooth gel.

Suitable pharmaceutically acceptable carriers include Hydroxypropylmethyl cellulose carbomer, white wax, canauba wax, anionic emulsifying wax, white petrolatum, polyethylene glycols, peanut oil and the like commonly used in pharmaceutical formulations.

### 7) EXAMPLE-1

Preparation of the Extracts of the Leaves and/or Stem of Argemone Mexicana Plant in Combination with the Extract of Fruits of Ciminum cyminum Plant [1] and the Lyophilized Powder [2]

Fresh leaves of Argemone mexicana Plant were collected and washed with water. 10-12 leaves (20.24 gm) were taken together with 10.09 gm of fruits of Cuminum cyminum plant and with 50 ml of water. This mixture was ground into a paste. The entire mixture was filtered through a muslin cloth. The filtrate, [1] can be administered as a single dose to the patients The extract can be lyophilized to yield 8.42% of [1] as a greenish-brown powder.

2.18 kg of fresh leaves of Argemone mexicana plant were ground with water, along with 1.149 kg of fruits of Cuminum cyminum plant. These were left for 30 minutes in 10 It water and then filtered through muslin cloth. The filtrate was, centrifuged and the filtrate was lyophilized to give 358 gm (16.4%) of greenish brown powder, [2].

### 8) EXAMPLE-II

Proof of Concept Study for the Extracts Obtained from the Leaves and/or stem of Argemone Mexicana Plant

The Psoriasis Area and Severity Index (PASI) score has persisted for a long time as a handy simplification to describe the severity of psoriasis. Clinically most investigators use the PASI score, which takes into account the total body surface area of lesional skin, as well as degree of erythrema, scaling and thickness to evaluate the efficacy of any given therapeutic protocol. The effectiveness of the composition according to present invention, for treatment of psoriasis was assessed by calculating PASI score during clinical tests every 2 weeks during the treatment regimen.

The clinical data shows excellent results obtained by using herbal composition in accordance with the present invention to treat mammals suffering from psoriasis.

A test population of 22 psoriatic patients who were classified as having chronic plaque type psoriasis was included in a study. The group included 19 males and 3 females. The patients were ranged from 25-75 years. The formulation [1] was given orally once daily for three consecutive days in a week for a maximum period of 12-15 weeks. PASI Score were recorded in the beginning as well as at every two weeks interval. No internal or topical treatment was applied during the period of therapy. About 50% of the patients showed 100% reduction of PASI score, about 50% of the patients showed 75-90% reduction in the PASI score and 1 patients showed about 56% reduction in the PASI score as compared to the initial score.

A statistical analysis of the data recorded during clinical trials on the 22 patients was undertaken to evaluate the efficacy and tolerability on the composition of the intervention and the method of treatment for psoriasis.

Mean PASI score of the patients recorded at every two weeks during the treatment regimen (Table-1) shows a graphical representation of the same, where ‘n’ stands for number of patients. A statistical reduction in the PASI score from 6.33±2.84 to 0.90±1.27 has been observed which is indicative that continuous administration of the herbal composition of the invention gives sustained reduction in the PASI score as treatment for psoriasis progresses. The disease free state was observed in some patients 8 weeks onwards.

TABLE 1
Actual PASI score (mean ± SD) during treatment duration

| Treatment duration | PASI score | Number of |
|---|---|---|
| (weeks) | (mean ± SD) | patients (n) |
| 0 | 6.33 ± 2.84 | 22 |
| 2 | 4.79 ± 2.16 | 22 |
| 4 | 3.53 ± 1.72 | 22 |
| 6 | 2.58 ± 1.58 | 22 |
| 8 | 1.86 ± 1.43 | 20 |
| 10 | 1.35 ± 0.91 | 17 |
| 12 | 0.94 ± 0.82 | 13 |
| 14 | 0.92 ± 0.90 | 6 |
| 15 | 0.90 ± 1.27 | 2 |

PASI score reduction was evident with herbal composition from second week onwards and every two weeks in comparison to basal PASI score (Table-2). The data indicates that uninterrupted administration of the herbal composition according to the method of treatment results in statistically significant rate of reduction in the PASI score. The PASI score declined in all the patients studied. The score declined from basal value of 6.33±2.84 (mean±SD) to post-treatment levels of 0.99±1.27 (P<0.001).

TABLE 2
Comparison of PASI score (mean ± SD) at various time
intervals during treatment duration

| Treatment | PASI score | PASI score | | |
|---|---|---|---|---|
| duration | before therapy | after therapy | | Number of |
| (Week) | (mean ± SD)(a) | (mean ± SD)(b) | (a) − (b) | patients (n) |
| 0-2 | 6.33 ± 2.84 | 4.79 ± 2.17 | 1.54 | 22 |
| 0-4 | 6.33 ± 2.84 | 3.53 ± 1.72 | 2.80 | 22 |
| 0-6 | 6.33 ± 2.84 | 2.58 ± 1.58 | 3.75 | 22 |
| 0-8 | 6.49 ± 2.91 | 1.86 ± 1.43 | 4.63 | 20 |
| 0-10 | 6.71 ± 2.88 | 1.35 ± 0.91 | 5.36 | 17 |
| 0-12 | 6.94 ± 2.28 | 0.94 ± 0.82 | 6.00 | 13 |
| 0-14 | 7.77 ± 2.23 | 0.92 ± 0.90 | 6.85 | 6 |
| 0-15 | 8.00 ± 0.42 | 0.90 ± 1.27 | 7.10 | 2 |

A graded system was used for analyzing the tolerability response to the treatment of the patients with the herbal composition during clinical trials (Table-3). The results proved excellent tolerability of the herbal composition in the treatment of psoriasis. No serious side effect was observed in any of the patients. There was no case of rebound, a condition where a patient's psoriasis becomes substantially more severe than baseline once treatment is completed.

TABLE 3
Tolerability studies

| | Number of patients (%) | | | | | | | |
|---|---|---|---|---|---|---|---|---|
| Grade/ | 2 | 4 | 6 | 8 | 10 | 12 | 14 | 15 |
| Week | (n = 22) | (n = 22) | (n = 22) | (n = 21) | (n = 18) | (n = 13) | (n = 6) | (n = 3) |
| Poor | 0 | 0 | 0 | 0 | 0 | 0 | 0 | 0 |
| Fair | 14 | 18 | 32 | 38 | 33 | 31 | 33 | 0 |
| Good | 73 | 77 | 68 | 62 | 67 | 62 | 33 | 100 |
| Excellent | 14 | 5 | 0 | 0 | 0 | 8 | 33 | 0 |

Where ‘n’ represents number of patients.

No abnormal changes, from the normal permissible changes, were observed with hematological, hepatic and renal functions during clinical trials.

### 9) EXAMPLE-III

### Preparation of Extract of Argemone Mexicana Plant [3]

Mature Argemone mexicana was collected during the flowering state. The leaves and/or stem of the fresh plant were segregated from the stalks, flowers, fruits and seeds. These were washed and ground into a paste.

30 Kg of the paste was placed in a vessel and mixed with 30 litres of water. The vessel was covered with a lid and allowed to stand for about 16 hrs at room temperature of about 23-27° C. At the end of percolation the valve was opened and extract collected. Another 30 litres of water was added to the residue. This was also percolated for about 16 hrs and collected as earlier. Both percolates were mixed together and filtered, centrifuged and decanted. The filtrate was reduced to one fifth of its volume on a rotary evaporator at 40° C. under vacuum. This extract was further lyophilized to give a greenish brown powder 2.1 Kg (7%), [3].

10) Biological Activities of the Various Extracts

Different enzyme inhibitions such as p70src Tyrosine kinase, p56lyn Tyrosine kinase, 5-Lipoxygenase, Phosphodiesterase (PDE) III, PDE IV, PDE V and Cyclin dependent kinase (CDK) I were performed at CEREP, USA. The cytokine assay, such as IL-2, IFNgamma, IL-10, lymphoproliferation inhibition such as ConA induced mice splenocytes proliferation inhibition, HDMEC proliferation, endothelial cell ICAM-1 expression, Keratinocyte proliferation and other in vivo activities such as MEST in mice, DTH in guinea pigs are described below in example III. The biological activities of different extracts are in given in Table-4.

TABLE 4
The Biological Activities of the various extracts of Argemone
mexicana Plant

| Sr. | | | | | |
|---|---|---|---|---|---|
| No. | Biological activities | Unit | [3] | [4] | [2] |
| 1. | p70src Tyrosine | % inhibition at | ND | ND | 99.8 |
| | kinase | 100 μg/ml | | | |
| 2. | p56 lyn Tyrosine | % inhibition at 50 | 9.5 | 28.6 | 48.0 |
| | kinase | μg/ml | | | |
| 3. | 5-Lipoxygenase | % inhibition at 100 | 46 | 100 | ND |
| | | μg/ml | | | |
| 4. | PDE III | % inhibition at 100 | 20 | 66 | ND |
| | | μg/ml | | | |
| 5. | PDE IV | % inhibition at 100 | 27 | 60 | ND |
| | | μg/ml | | | |
| 6. | PDE V | % inhibition at 100 | 35 | 75 | ND |
| | | μg/ml | | | |
| 7. | CDK1 | % inhibition at 100 | 1 | 57 | ND |
| | | μg/ml | | | |
| 8. | IL-10 | % induction from | 235 | 116 | 166 |
| | | basal at 50 μg/ml | | | |
| 9. | IFN gamma | % inhibition at 50 | 61 | 27 | 51 |
| | | μg/ml | | | |
| 10. | IL-2 | % inhibition at 50 | 57 | 24 | 45 |
| | | μg/ml | | | |
| 11. | Immunosuppression | ED₅₀ in mg/kg | 14 | ND | 238 |
| | in MEST model | | | | |
| 12. | Delayed type hyper- | % inhibition at human | 83 | 12 | 52 |
| | sensitivity in | equivalent dose | | | |
| | Guinea pigs | | | | |
| 13. | ConA induced mice | IC₅₀ in μg/ml | 78 | 229 | 184 |
| | splenocytes | | | | |
| | proliferation | | | | |
| | inhibition | | | | |
| 14. | NGF induced | % inhibition at 12 | 70 | 20 | ND |
| | HDMEC prolifer- | ng/ml | | | |
| | ation inhibition | | | | |
| 15. | NGF induced Endo- | % inhibition at 12 | 51 | 23 | ND |
| | thelial ICAM-1 | ng/ml | | | |
| | expression | | | | |
| 16. | NGF induced | % inhibition at 12 | 76 | 31 | ND |
| | Keratinocytes | ng/ml | | | |
| | proliferation | | | | |

ND = not done

The aqueous extract of Argemone mexicana was further investigated for accessing IC₅₀ value and found to be inhibitory to p60src Tyrosine kinase with IC₅₀ of 64.15 μg/ml. Inhibitors of protein tyrosine kinases (PTKs) play crucial role in anti-psoriatic treatment. PTKs are closely associated with cell growth, proliferation, differentiation and signaling of the immune systems. The PTK inhibitor property is useful in inhibiting lymphocyte, keratinocytes and endothelial cells receptor signaling transduction for proliferation, differentiation and function, further comprising phosphorylation of key signaling molecules.

Inhibition of 5-Lipoxygenase in Arachidonic acid pathway with aqueous extract of Cuminum cyminum [41 proved the anti-inflammatory activities in different inflammatory diseases.

### 11) EXAMPLE-IV

Preparation of Extract Obtained from the Fruits of Cuminum Cyminum Plant [4]

27.5 kg of fruits of Cuminum cyminum were taken and ground into a powder. 82.5 litres of water was added. The vessel was covered with a lid and allowed to stand for about 16 hrs at room temperature of about 23-27° C. At the end of percolation the valve was opened and extract collected. The percolate was filtered, centrifuged and decanted. The filtrate was reduced to one fifth of its volume on a rotary evaporator at 40° C. under vacuum. This extract was further lyophilized to give a brown powder 2.38 kg (8.65%), 14].

### 12) EXAMPLE-V

Preparation of n-Butanol-Soluble Fraction [5], Methanol-Soluble Fraction [6] and Methanol-Insoluble Fraction of the Extracts of the Leaves and/or Stem of Argemone Mexicana Plant [7]

25 gm of aqueous extract of the leaves of Argemone mexicana Plant was dissolved in 375 ml of water. This combination was stirred, centrifuged and filtered. Filtrate was fractionated with 3×250 ml with n-butanol. The butanol layer was washed with 125 ml of water, concentrated and dried under vacuum at 40° C. to give 1.5 gm (5.97%) of the n-butanol-soluble fraction, [5].

The aqueous fraction was poured in 2.5 litres of methanol with continuous stirring. After precipitation the solution was centrifuged and supernatant was decanted. This methanol soluble fraction was concentrated under vacuum at 40° C. and lyophilized to give 12.46 gm (49.8%) of the methanol-soluble fraction, [6].

The methanol-insoluble precipitate was dried under vacuum at 40° C. and lyophilized to give 8.48 gm (33.89%) of a solid, which was dissolved in 85 ml water, sonicated, centrifuged and filtered. The filtrate was lyophilized to give 6.67 gm (26.7%)of methanol-insoluble and water-soluble fraction, [7].

12) Biological Activities of the Fractions

12-1: Description of the Methods of Evaluation for Mitogen Induced Lymphoproliferation Inhibition

In order to evaluate the efficacy of the fractions of Argemone mexicana for its therapeutic potential in psoriasis, its role as an immunosuppressent was evaluated in in vitro mitogen induced lymphocyte proliferation inhibition assay. Mitogen induced lymphoproliferation inhibition (Gougerot-Pocidalo, M. A. et al., 1988; Dayton, J. S., et al., 1992) was employed for evaluation of in vitro immunosuppressive property.

Briefly, C57 mice splenocytes were separated. One million/ml splenocytes were stimulated with ConA (10 μg/ml) along with various concentrations of different extracts/fractions of Argemone mexicana, Cuminum cyminum and formulation for 5 days at 37° C. in CO₂ incubator with 5% CO₂. The proliferating cells were enumerated with MTT assay.

The effect of extracts and fractions of Argemone mexicana, [3], [5], [6,], and [7] on ConA induced mice splenocytes proliferation, in IC₅₀ in μg/ml are:

The aqueous extract [3], showed ConA induced lymphoproliferation inhibition with IC₅₀ of 78 μg/ml; n-butanol extract [5] with IC₅₀ of 34 μg/ml where as methanol soluble extract [6] with >200 μg/ml and methanol insoluble extract [7] with >80 μg/ml.

n-butanol extracts [5] of Argemone mexicana were found inhibitory to ConA induced proliferation of mice splenocytes. This inhibitory activity to mitogen-induced lympho-proliferation is known to be immunosuppressive and well established to be useful in treatment of psoriasis.

The invention includes lymphocyte proliferation inhibition activity, which includes CD4+, CD8+ cells. These are related to immunosuppression which help in treating psoriasis, dermatitis, scleroderma, inflammatory disorders and other autoimmune diseases like psoriatic arthritis, plaque psoriasis, guttate psoriasis, rheumatoid arthritis, Crohn's disease, multiple sclerosis, ankylosing spondilitis, systemic lupus erythremetosus, Sjogren's syndrome, allergies like asthma, chronic obstructive pulmonary disease and related conditions as eczema, scaly itchy patches. Immunosuppression is also related to various organ transplants.

12-2: Description of the Methods of Evaluation for Human Keratinocyte Proliferation Inhibition

In order to evaluate the efficacy of the fractions of Argemone mexicana for its therapeutic potential in psoriasis, its role in in vitro human keratinocyte proliferation inhibition was evaluated by Hexosamimidase assay (Raychudhuri, S. K., et al., 2001).

Briefly, human keratinocyte were separated from human skin biopsies, and added 2000 cells/well in a 96 well flat bottom plate in 150 μl keratinocyte growth medium. Next day medium was changed with 150 μl of keratinocyte growth medium: keratinocyte basal medium (1:2 volume). 100 ng/ml of NGF was added as growth factor. Serial dilution of different extracts at various concentrations was added in quadruplicate wells. Four wells were kept with medium only as baseline value. After 4,6 and 8 days culture at 37° C. in a humidified, 5% CO₂ incubator, remove medium, rinse cells with PBS. 60 μl of Substrate (7.5 nM p-nitrophenyl-N-acetyl-b-D-glycosaminide in 0.1M citrate buffer, pH 5.0. The solution is then mixed with equal volume of 0.5% Triton X-100 in water, aliquoted and stored at −20° C.) was added to each well and incubated at 37° C. in a humidified, 5% CO₂ incubator for 90 min. 90 μl of the stop solution (50 mM glycine buffer, pH 10.4 containing 5 mM EDTA). 125 μl of supernatant was transferred to 96 well plates for absorbance reading at 405 nm.

TABLE 5(A)
Effect of different fractions of Argemone mexicana
Plant on human keratinocyte proliferation.
% inhibition of human keratinocyte proliferation

| Concentration of | | | |
|---|---|---|---|
| ext. (ng/ml) | [5] | [6] | [7] |
| 320 | −5.3 | 31.6 | 47.4 |
| 64 | 13.8 | 38.5 | 67.7 |
| 12 | −10 | 24 | 44 |
| 2.5 | 5.7 | 20.8 | 24.5 |
| 0.48 | 2 | 5.9 | −2 |

* The values are depicted in percentage inhibition with reference to control.

TABLE 5(B)
Effect of different fractions of Argemone mexicana Plant
on NGF induced human keratinocyte proliferation.
% inhibition of NGF induced keratinocyte proliferation

| Concentration of | | | |
|---|---|---|---|
| ext. (ng/ml) | [5] | [6] | [7] |
| 320 | 59.4 | 69.3 | 60.4 |
| 64 | 34 | 70 | 31 |
| 12 | 39.6 | 75.2 | 41.6 |
| 2.5 | 36.7 | 24.2 | 22.7 |
| 0.48 | 0 | 7.1 | 8.6 |

* The values are depicted in percentage inhibition with reference to control.

Different extracts and fractions of Argemone mexicana plant were found inhibitory to human keratinocyte proliferation and NGF induced proliferation in range of 320 ng/ml to 2.5 ng/ml. This antiproliferative property to keratinocytes is useful for reducing hyperplasia of epidermis and well established to be useful in treatment of psoriasis.

The invention relates to human keratinocyte proliferation and NGF induced proliferation inhibition which is useful in disease where keratolytic activity is required such as psoriasis, psoriatic arthritis, plaque type psoriasis, guttate psoriasis, pustular psoriasis, psoriasis with silvery scale and other skin ailments including epidermal hyperplasia, impaired cell mediated immunity resulting in increased infections to skin, involvement of proliferation marker such as Ki67 and PCNA, chronic skin ailment, recurrent skin ailment, hyperplasia of keratinocytes, apoptotic resistant keratinocytes, discrete erthymatosus papules and scaly plaques.

12-3: Description of the Methods of Evaluation for Human IL-2 and IFN Gamma Production

In order to evaluate the efficacy of fractions of Argemone mexicana plant for its therapeutic potential in psoriasis, its role in in vitro IL-2 and IFN gamma inhibition was evaluated by PHA induced PBMCs IL-2 and IFN gamma assay (Brynskov, J., et al, 1990; Ho, L. J., et al., 1999).

The aim is to study the effect of extract on PHA induced IL-2 and IFNgamma production from human lymphocyte. Briefly, peripheral blood mononuclear cells (PBMC) were obtained from healthy individuals. One million PBMC from each volunteer were stimulated with PHA (5 μg/ml) along with various concentrations of different extract of Argemone mexicana for 48 hours at 37° C. in CO₂ incubator with 5% CO₂. Supernatant were harvested and frozen at −70° C. Human IL-2 and Human IFN gamma ELISA kit were used from BD Pharmingen for detection of IL-2 and IFN gamma in culture supernatant. Percent inhibition was calculated with reference to control.

TABLE 6(A)
Effect of different fractions of Argemone mexicana Plant
on PHA induced human PBMCs IL-2 production.
% inhibition of PHA induced IL-2 production from
Human PBMCs

| Concentration | | | |
|---|---|---|---|
| of ext. (ng/ml) | [5] | [6] | [7] |
| 64 | 5.3 | 57.9 | 65.8 |
| 12 | 5.1 | 49.7 | 54.8 |
| 2.5 | 5.3 | 10.1 | 11.2 |
| 0.48 | −4.7 | 1.3 | −2.6 |

* The values are depicted in percentage inhibition with reference to control.

TABLE 6(B)
Effect of different fractions of Argemone mexicana Plant
on NGF induced human PBMCs IL-2 production.
% inhibition of NGF induced IL-2 production from Human
PBMCs

| Concentration | | | |
|---|---|---|---|
| of ext. (ng/ml) | [5] | [6] | [7] |
| 64 | 23.5 | 58.8 | 17.6 |
| 12 | 24.2 | 57.9 | 29.5 |
| 2.5 | 35.3 | 18.2 | 17.6 |
| 0.48 | 5 | 11.8 | 15 |

* The values are depicted in percentage inhibition with reference to control.

Different fractions of Argemone mexicana were found inhibitory to mitogen induced IL-2 production and NGF induced IL-2 production in range of 64 ng/ml to 2.5 ng/ml. This inhibitory activity to mitogen/NGF induced IL-2 production is known to be immunosuppressive and well established to be useful in treatment of psoriasis.

TABLE 7(A)
Effect of different fractions of Argemone mexicana Plant
on PHA induced human PBMCs IFN gamma production.
% inhibition of PHA induced IFNgamma production from
Human PBMCs

| Concentration | | | |
|---|---|---|---|
| of ext. (ng/ml) | [5] | [6] | [7] |
| 64 | 25 | 11 | 10 |
| 12 | 25 | 11 | 10 |
| 2.5 | 11.9 | 8 | 5 |
| 0.48 | 0.9 | 1 | 0.5 |

* The values are depicted in percentage inhibition with reference to control.

TABLE 7(B)
Effect of different fractions of Argemone mexicana Plant on
NGF induced human PBMCs IFN gamma production.
% inhibition of NGF induced IFN gamma production from
Human PBMCs

| Concentration | | | |
|---|---|---|---|
| of ext. (ng/ml) | [5] | [6] | [7] |
| 64 | 45 | 35.1 | 41 |
| 12 | 45 | 35 | 40.9 |
| 2.5 | 4.1 | 4.4 | 7.1 |
| 0.48 | 5 | 11.8 | 15 |

* The values are depicted in percentage inhibition with reference to control.

Different fractions of Argemone mexicana were found inhibitory to mitogen induced IFN gamma inhibition and NGF induced IFN gamma inhibition in range of 64 ng/ml to 2.5 ng/ml. This inhibitory activity to mitogen/NFG induced IFN gamma production is known to be immunosuppressive and well established to be useful in treatment of psoriasis.

The invention relates to IL-2 and IFN gamma production inhibition which is useful in disease wherein excessive TH1 cytokine involvement is present such as psoriasis, dermatitis, scleroderma, inflammatory disorders and other autoimmune diseases like psoriatic arthritis, plaque psoriasis, guttate psoriasis, rheumatoid arthritis, Crohn's disease, multiple sclerosis, irritable bowel disease, ankylosing spondilitis, systemic lupus erythremetosus, Sjogren's syndrome, allergies like asthma, chronic obstructive pulmonary disease and related conditions as eczema, scaly itchy patches. IL-2 and IFN gamma inhibition is also useful in various organ transplants.

12-4: Description of the Methods of Evaluation for Human IL-10 Production

In order to evaluate the efficacy of the extracts and fractions of Argemone mexicana for its therapeutic potential in psoriasis, its role in in vitro IL-10 induction was evaluated by ConA induced PBMCs IL-10 production assay (Raychaudhuri, S. P., et al., 1999).

Briefly, Human PBMCs were separated out and stimulated with 10 μg/ml ConA along with various concentrations of different extract of Argemone mexicana and incubated for 48 hours at 37° C. in CO₂ incubator with 5% CO₂. Supernatant were harvested and frozen at −70° C. Human IL-10 ELISA kit were used from BD Pharmingen for detection of IL-10 in culture supernatant. Percent induction was calculated with reference to control.

TABLE 8
Effect of different fractions of Argemone mexicana Plant on
ConA activated human PBMCs IL-10 production.
Percent increase (from basal) of ConA induced IL-10
production. from Human PBMCs

| Concentration | | | |
|---|---|---|---|
| of ext. (μg/ml) | [5] | [6] | [7] |
| 200 | 1.7 | 17.7 | 171.1 |
| 20 | 0.5 | 14.0 | 162.1 |
| 2 | 1.0 | 5.5 | 98.3 |
| 0.2 | −0.5 | 1.0 | 24.6 |
| 0.02 | −2.0 | 0.0 | −4.0 |
| 0.002 | −4.0 | −2.0 | −5.0 |

* The values are depicted in percentage increase from basal with reference to control.

Aqueous extract and methanol insoluble extracts of Argemone mexicana were found to be inducer for IL-10 in ConA activated human PBMCs in the range of 200 μg/ml to 0.2 μg/ml. IL-10 was found to be regulatory cytokine in psoriasis treatment and well established cytokine for anti-psoriatic therapy.

IL-10 induction is useful in psoriasis, dermatitis, scleroderma, inflammatory disorders and other autoimmune diseases like psoriatic arthritis, plaque psoriasis, guttate psoriasis, rheumatoid arthritis, Crohn's disease, multiple sclerosis, irritable bowel disease, ankylosing spondilitis, systemic lupus erythremetosus, Sjogren's syndrome, allergies like asthma, chronic obstructive pulmonary disease and related conditions as eczema, scaly itchy patches. IL-10 induction is also useful in other chronic, recurrent and other skin ailments where cutaneous lymphocyte antigen or cutaneous leukocyte antigen is required.

12-5: Description of the Methods of Evaluation For human HDMEC—ICAM-1 Expression Inhibition

In order to evaluate the efficacy of the different fractions of Argemone mexicana for its therapeutic potential in psoriasis, its role in in vitro human endothelial cell ICAM-1 expression was evaluated by ELISA.(Raychudhuri, S. P., et al., 2001).

Briefly, HDMEC (2×10⁴ cells/well) were plated in 24 well plates pretreated with attachment factor for 24 h. Media (EGM-MV) contains 20% fetal calf serum (FCS). Each experiment was done in triplicate. After 24 h, media was changed to EGM-MV containing 5% FCS and the following reagents were added separately, NGF-β 100 ng/ml), NGF-β with anti-NGF antibody along with various concentrations of different extracts of Argemone mexicana. Three wells were kept with medium only as control. After 24 h incubation, cells were washed three times with PBS. Cells were fixed with 0.5% gluteraldehyde for 10 min. At 4° C., cells were washed three times with PBS+5 mM EDTA+0.1% bovine serum albumin (BSA). Then ICAM-1 monoclonal antibody at 0.5 μg/ml concentration was added to the wells. After 1 h, the wells were rinsed three times with PBS+0.1% BSA and 100 μl/well goat anti-mouse IgG antibody labeled with peroxidase (1:1000 in HBSS=0.1%BSA) was added. After 1 h, wells were washed and TMB solution (100 μl/well) was added to each well. After 30 minute H₂SO₄ (0.5 M, 50 μl/well) was added and mixed. One hundred microliter of the mixture was transferred to 96 well plates and read at 492 nm. Percent induction was calculated with reference to control.

TABLE 9(A)
Effect of different fractions of Argemone mexicana Plant on
LPS induced ICAM-1 expression on human dermal
microvascular endothelial cells (HDMEC).
% inhibition of LPS induced ICAM-1expression from HDMEC

| Concentration | | | |
|---|---|---|---|
| of ext. (ng/ml) | [5] | [6] | [7] |
| 320 | 5.5 | 11.7 | 51.6 |
| 64 | 6.7 | 12 | 52 |
| 12 | 5.1 | 52 | 52.5 |

* The values are depicted in percentage inhibition with reference to control.

TABLE 9(B)
Effect of different fractions of Argemone mexicana Plant on
NGF induced ICAM-1 expression on human dermal microvascular
endothelial cells (HDMEC).
% inhibition of NGF induced ICAM-1expression from HDMEC

| Concentration | | | |
|---|---|---|---|
| of ext. (ng/ml) | [5] | [6] | [7] |
| 320 | 13.3 | 33.1 | 31.9 |
| 64 | 14 | 33.3 | 29.8 |
| 12 | 14.3 | 33.3 | 12.9 |

* The values are depicted in percentage inhibition with reference to control.

Different extracts and fractions of Argemone mexicana plant were found inhibitory ICAM-1 expression on LPS or NGF induced HDMEC in the range of 320 ng/ml to 2.5 ng/ml. This ICAM-1 inhibition is well known for affecting lymphocyte and monocyte trafficking to the skin lesion site and potential for treating psoriasis conditions.

The invention is useful in the disease wherein cell adhesion inhibition, cell adhesion expression inhibition, integrin inhibition are required such as immune cell trafficking, lymphocyte trafficking, monocyte trafficking, neutrophils trafficking, macrophages trafficking. This cell adhesion inhibition will be useful scleroderma, inflammatory disorders and other autoimmune diseases like psoriatic arthritis, plaque psoriasis, guttate psoriasis, rheumatoid arthritis, Crohn's disease, multiple sclerosis, irritable bowel disease, ankylosing spondilitis, systemic lupus erythremetosus, Sjogren's syndrome, allergies like asthma, chronic obstructive pulmonary disease and related conditions as eczema, scaly itchy patches.

12-6: Description of the Methods of Evaluation for Human HDMEC Proliferation Inhibition

In order to evaluate the efficacy of different fractions of Argemone mexicana for its therapeutic potential in psoriasis, its role in in vitro human endothelial cell proliferation was evaluated by Hexosamimidase assay.(Raychudhuri, S. P., et al., 2001).

Briefly, Adult HDMEC from pooled donors was used in third passage. The culture medium was EGM-MV without hydrocortisone. Endothelial cell proliferation was assessed by Hexosamimidase assay. The substrate for Hexosamimidase was p-nitrophenol-N-acetyl-β-D-glucosaminide. NGF-β was used as stimulant. NGF-β was used in 100 ng/ml. NGF-neutralizing antibody (100 ng/ml) was used to asses the inhibition of NGF-induced EC proliferation.

3300 cells per well were plated in flat bottom 96 well microtiter plates. After 24 h NGF-β and NGF+NGF-neutralizing antibody were added for activating the cells in triplicate wells. The cells were incubated for 2 and 5 days. The substrate solution was added in volumes of 60 μl to cells in flat bottom microtiter wells. The plates were then incubated at 37° C. in 100% humidity. After a suitable interval, the color reaction was developed and enzyme activity blocked by addition of 50 mM glycine buffer, pH10.4, containing 5 mM EDTA and 90 μl per well. Absorbance was measured in an ELISA reader at 405 nm.

TABLE 10(A)
Effect of different fractions of Argemone mexicana Plant on
human dermal microvascular endothelial cells (HDMEC)
proliferation.
% inhibition of HDMEC proliferation

| Concentration | | | |
|---|---|---|---|
| of ext. (ng/ml) | [5] | [6] | [7] |
| 320 | 2 | 18.4 | 38.8 |
| 64 | 3.3 | 18.3 | 18.3 |
| 12 | 2.1 | 18.8 | 18.8 |

* The values are depicted in percentage inhibition with reference to control.

TABLE 10(B)
Effect of different fractions of Argemone mexicana Plant on
NGF induced human dermal microvascular endothelial cells
(HDMEC) proliferation.
% inhibition of NGF induced HDMEC proliferation

| Concentration | | | |
|---|---|---|---|
| of ext. (ng/ml) | [5] | [6] | [7] |
| 320 | 20 | 45.5 | 20.9 |
| 64 | 12 | 40 | 14 |
| 12 | 20 | 45 | 21.4 |

* The values are depicted in percentage inhibition with reference to control.

Different extracts and fractions of Argemone mexicana were found inhibitory to human dermal microvascular endothelial cells (HDMEC) proliferation with and without NGF in range of 320 ng/ml to 12 ng/ml. This antiproliferative property to human dermal microvascular endothelial cells (HDMEC) is useful for reducing angiogenesis of dermis and well established to be useful in treatment of psoriasis

The invention is useful in the disease wherein angiogenesis inhibition is required such as psoriasis and cancer.

12-7: Description of the Methods of Evaluation of In Vivo Immunosuppression using Mouse Ear Swelling Test

This standard procedure was used for evaluation of the in vivo efficacy of different extracts and fractions of Argemone mexicana for their ability to inhibit DNFB induced delayed type hypersensitivity in mice(Cornacoff, J. B., et al.,1992; Cornacoff, J. B., et al., 1988).

Briefly, C57BL6 mice were used for the test. Mice were sensitized with 0.2% DNFB (in 1:4 of Olive oil and Acetone) on back of the mice. Three boosters DNFB application were done at every third day. The mice were challenged with 0.2% DNFB (in 1:4 of Olive oil and Acetone) on ear pinna. Ear thickness was taken in a center of the ear with the help of Varnier caliper after 24 hours. Analysis was performed, by calculating percent inhibition with respect to negative control. Extract and fraction were given orally at different doses.

The aqueous[3] and methanol-insoluble extracts[7] of Argemone mexicana were found to be immunosuppressive to DNFB sensitized C57BL6 mice. The ED₅₀ for aqueous extract [3] was determined to be 13.7 mg/kg while for a methanol-insoluble extract [7] was 44.87 mg/kg. The potent immunosuppressive property is well established and beneficial for anti-psoriasis treatment.

The invention like immunosuppression in MEST model is useful in several diseases where immunosuppression is required such as psoriasis, dermatitis, scleroderma, inflammatory disorders and other autoimmune diseases like psoriatic arthritis, plaque psoriasis, guttate psoriasis, rheumatoid arthritis, Crohn's disease, multiple sclerosis, ankylosing spondilitis, systemic lupus erythremetosus, Sjogren's syndrome, allergies like asthma, chronic obstructive pulmonary disease and related conditions as eczema, scaly itchy patches. Immunosuppression is also useful in various organ transplants.

12-8: Description of the Methods of Evaluation of In Vivo Immunosuppression using Delayed Type Hypersensitivity in Guinea pigs

This standard procedure was used for evaluation of the in vivo efficacy of different extracts for their ability to inhibit Purified protein derivative (PPD) induced delayed type hypersensitivity in guinea pigs. Briefly, guinea pigs were sensitized with 100 μg of PPD intradermally with Freund's complete adjuvant. Two subsequent boosters of 100 μg of PPD with Freund's incomplete adjuvant were at a week interval. Extract and fractions were given orally at different doses. The animals were challenged with 100 μg of PPD intradermally and skin thickness was recorded with Varnier's calipers. The differences of skin thickness were calculated by subtracting saline injected skin thickness in same guinea pigs. Percent inhibition was calculated with reference to saline sensitized animals.

The aqueous extract of Argemone mexicana [3] and Cuminum cyminum [4] were found to be immunosuppressive to PPD sensitized and PPD challenged guinea pigs in the range of 52-85% inhibition. The potent immunosuppressive property is well established and beneficial for anti-psoriasis treatment.

The invention like immunosuppression in DTH model is useful in several diseases where immunosuppression is required such as psoriasis, dermatitis, scleroderma, inflammatory disorders and other autoimmune diseases like psoriatic arthritis, plaque psoriasis, guttate psoriasis, rheumatoid arthritis, Crohn's disease, multiple sclerosis, ankylosing spondilitis, systemic lupus erythremetosus, Sjogren's syndrome, allergies like asthma, chronic obstructive pulmonary disease and related conditions as eczema, scaly itchy patches. Immunosuppression is also useful in various organ transplants.

### REFERENCES

Alice B. Gottlieb, Medical Dermatology, 2001, 19(4), 649.

Ashcroft D. M., Wan Po A. L., Williams H. C. and Griffiths C. E. M., BMJ, 2000, 320, 963.

Asprey, G. F., and Thornton, P., West Indian Med. J., 1955, 4, 145.

Austin L. M, Ozawa M, Kikuchi T, Walters I. B, Krueger J. G., J. Invest. Dermatol., 1999, 13, 752.

Bata-Csorgo Z, Hammerberg C, Voorhees J. J, Cooper K. D., J. exp Med., 1993, 178, 1271.

Baumann G, Zenke G, Wenger R, Hiestand P, Quesniaux V, Andersen E, Schreier M. H., J Autoimmun., 1992, 5 Suppl A, 67.

Beutner E. H., Autoimmunity in Psoriasis, CRC Press, 1982.

Bialasiewicz A. A, Lubach D, Marghescu S., Arch Dermatol Res., 1981, 271(1), 29.

Bloemena E, van Oers M. H, Weinreich S, Yong S. L, Schellekens P. T., Clin. Immunol. Immunopathol., 1988, 48(3), 380.

Boffa M. J., Adv Exp Med Biol., 1999, 455, 341.

Boss J. D., Clin. Exp. Immunol., 1997, 107(Suppl 1), 3.

British National Formulary (BNF), March 2001, No. 41.

Brynskov J, Tvede N., Gut., 1990, 31(7), 795.

Cornacoff J. B, House R. V, Dean J. H., Fundam. Appl. Toxicol., 1988, 10(1), 40.

Cornacoff J. B, House R. V, Dean J. H. Fundam. Appl. Toxicol., 1992, 19(1), 157. Creamer D, Allen M. H., Sousa A, Poston R, Barker J. N. Br. J. Dermatol., 1997, 136(6), 859.

Dayton J. S., Turka L. A., Thompson C. B., Mitchell B. S., Mol. Pharmacol., 1992, 41,671.

Duke, J. A., Handbook of Medicinal Herbs, 59, Pub. By CRC Press, 2001.

Duncan J. I., J. Invest. Dermatol., 1994, 102(1), 84.

Dupont E, Schandene L, Denys C, Wybran J., Clin Immunol Immunopathol., 1986, 40 (3), 422.

Dustin M. L., Springer T. A., J. Cell. Biol., 1988, 107(1), 321.

Elridge, J., Econ. Bot., 1975, 29, 307.

Feldman S. R., American Academy of Dermatology, August 2000.

Finzi A. F., Mozzanica N, Pigatto P. D., Chiappino G., Acta. Derm. Venereol. Suppl. (Stockh), 1989, 146, 137.

Flanagan W. M., Crabtree G.R., Ann N.Y., Acad. Sci., 1993, 696, 31.

Fortune D. G., Richards H. L., Main C. J., and Griffiths C. E. M., J. Am. Acad. Dermatol., 1998, 39,196.

Gelfand E. W., Cheung R. K., Mills G. B., J. Immunol., 1987, 138(4), 1115.

Gougerot-Pocidalo M. A., Fay M, Roche Y, Chollet-Martin S., Immunology, 1988, 64, 281.

Griffiths C. E, Vorhees J. J, Nickoloff B. J., J. Am. Acad. Dermatol., 1989, 20(4), 617.

Gurib-Fakim, A., Sweraj, M. D., Gueho, J. and Dulloo E., Int. J. Pharmacog., 1996, 34, 2.

Halberstein, R. A., and Saunders, A. B., Cul. Med. Psychiat., 1978, 2, 177.

Hannah Ben-Bassat, Current Opinion in Investigational Drugs, 2001, 2(11), 1539.

Henderson D. J., Naya I, Bundick R. V., Smith G. M., Schmidt J. A., Immunology, 1991, 73(3),316.

Ho L. J., Chang D. M., Chang M. L., Kuo S. Y., Lai J. H., J. Rheumatol., 1999, 26(1), 14.

Hunter T, Alexander C. B., Cooper J. A., Ciba Found. Symp., 1985, 116, 188.

Inohara S., Int. J. Dermatol., 1992, 31, 237.

Khusru Asadullah, Wolfram S, Katja S, Dominik J, Mattias L, Heike A, Hans-Dieter V, Wolf-Dietrich D., J. Clin. Invest., 1998, 101(4), 783, 1998.

Kirtikar, K. R., and Basu, B. D., Indian Medicinal Plants, Vol. 1, 2^{nd} Ed, Ed. Blatter, E., Caius, J. F., and Mhaskar, K. S., Pub. By Basu, L. M., 1933.

Kragballe K., Arch. Dermatol. Res., 1992, 284 Suppl 1, S30.

Kuroda K, Sapadin A, Shoji T, Fleischmajer R, Lebwohl M., J. Invest. Dermatol., 2001, 116(5),713.

Levantine A, Brostoff J., Br. J. Dermatol., 1975, 93(6), 659.

McKay I. A., Leigh I. M., Clin. Dermatol., 1995, 13, 105.

Mehta R. C., Stecker K. K., Cooper S. R., Templin M. V., Tsai Y. J., Condon T. P., Bennett C. F., Hardee G. E., J. Invest. Dermatol., 2000, 115(5), 805.

Mollison K. W., Fey T. A., Gauvin D. M., Sheets M. P., Smith M. L., Pong M, Krause R, Miller L, Or Y. S., Kawai M, Wagner R, Wiedeman P. E., Clark R. F., Gunawardana I. W., Rhoades T. A., Henry C. L., Tu N. P., BaMaung N. Y., Kopecka H, Liu L, Xie Q, Lane B. C., Trevillyan J. M., Marsh K, Luly J. R., Curr. Pharm. Des., 1998, 4(5), 367.

Oakes, A. J., and Morris, M. P., Bull. Hist. Med., 1958, 32, 164.

Munro J. M., Pober J. S., Cotran R. S., Lab. Invest., 1991, 64, 295.

Munro J. M., Pober J. S., Cotran R. S., Am. J. Pathol., 1989, 135, 121.

Nickoloff B. J., Arch. Dermatol., 1991, 127, 871.

Oppenheimer-Marks N, Davis L. S., Tompkins-Bogue D, Ramberg J, Lipsky P. E., J. Immunol., 1991, 147, 2913.

Platzer C, Meisel C, Vogt K, Platzer M, Volk H. D., Int. Immunol., 1995, 7, 517.

Rachudhuri S. K., Raychudhuri S. P., Weltman H and Farber E. M., Arch. Dermatol. Res., 293, 291.

Raychaudhuri S. K., Raychaudhuri S. P., Weltman H, Farber E. M., Arch. Dermatol. Res., 2001, 293(6), 291.

Raychudhuri S. P., Farber E. M., Raychudhuri S. K., Int. J. Immunopharmacol., 1999, 21,609.

Reich K., Garbe C., Blaschke V., Maurer C., Middel P., Westhal G., Lippert U. and Neumann C., J. Invest. Dermatol., 2001, 116, 319.

Siekierka J. J., Immunol. Res., 1994, 13(2-3), 110.

Siemeister G, Schirner M, Reusch P, Barleon B, Marme D, Martiny-Baron G., Proc. Natl. Acad. Sci. U.S.A., 1998, 14;95(8):4625.

Stern R. S., and Laird N., Cancer, 1994, 73, 2759.

Thomson A. W., Immunol. Lett., 1991, 29(1-2), 105.

Ullrich A, Schlessinger J, Cell, 1990, 61,203.

Uyemura K., Yamamura M., Fivenson D. F., Modlin R. L., Nickoloff B. J., J. Invest. Dermatol., 1993, 101, 701.

Van Ruissen F, de Jongh G. J., van Erp P. E., Boezeman J. B., Schalkwijk J. J., Cell. Physiol., 1996, 168, 684.

Wakita H, Takigawa M, Arch. Dermatol., 1994, 130, 457.

1. A herbal composition, for oral administration and topical application for the treatment of psoriasis and related biochemical and immunological disorders, comprising an effective amount of, an extract of the leaves and/or stem of Argemone mexicana plant, containing organic constituents, in combination with pharmaceutically acceptable carriers, the said extract exhibiting interesting immunological and pharmacological properties.

2. A herbal composition, for oral administration for the treatment of psoriasis and related biochemical and immunological disorders comprising an effective amount of an extract of the leaves and/or stem of Argemone mexicana plant, containing organic constituents, and an extract of the fruits of Cuminum cyminum plant in combination with pharmaceutically acceptable carriers, the said extract exhibiting interesting immunological and pharmacological properties

3. A method of treatment of psoriasis and related biochemical and immunological disorders comprising administration to a mammal the herbal composition of claim 1.

4. A method of treatment of psoriasis and related biochemical and immunological disorders comprising administration to a mammal the herbal composition of claim 2.

5. A herbal composition according to claim 1 wherein the extract of the leaves and/or stem of Argemone mexicana plant is a water extract, an ethanolic extract or a water-ethanolic extract and is in the liquid form.

6. A herbal composition according to claim 1, wherein the extract of the leaves and/or stem of Argemone mexicana plant is a dry powder.

7. A herbal composition according to claim 2, wherein the extract of the fruits of Cuminum cyminum plant is a water extract, an ethanolic extract or a water-ethanolic extract and is in the liquid form.

8. A herbal composition according to claim 2, wherein the extract of the fruits of Cuminum cyminum plant is a dry powder.

9. A herbal composition according to claims 1 wherein the organic constituents present in the extract of the leaves and/or stem of Argemone mexicana plant include a mixture of alkaloids, flavonoids, organic acids, amino acids, free sugars/glycosides and salts.

10. A herbal composition, for oral administration, according to claim 1, wherein the amount of the extract of the leaves and/or stem of Argemone mexicana plant comprises from 50 to 5000 mg by weight of the herbal composition,

11. A herbal composition, for oral administration, according to claim 2, wherein the extract of the leaves and/or stem of Argemone mexicana plant comprises 10 to 50% by weight of the herbal composition, the balance consisting of the extract of the fruits of Cuminum cyminum plant and/or pharmaceutically acceptable carriers.

12. A herbal composition, for oral administration, according to claim 2, comprising of 10 to 50% by weight of an extract from the leaves and/or stem of Argemone mexicana plant and from 60 to 90% by weight of an extract from the fruits of Cuminum cyminum plant, the balance, if any, consisting of pharmaceutically acceptable carriers.

13. A herbal composition, for oral administration, according to claim 2, wherein the amount of the extract of the leaves and/or stem of Argemone mexicana plant comprises from 2 to 100 mg by weight of the herbal composition, the balance consisting of the extract from the fruits of Cuminum cyminum plant and/or pharmaceutically acceptable carriers.

14. A herbal composition, for topical application, according to claim 1, wherein the extract of the leaves and/or stem of Argemone mexicana plant comprises 0.5% to 10% by weight of the extract.

15. A method as claimed in claim 3 wherein said related biochemical and immunological disorders include skin ailments, inflammatory disorders, autoimmune diseases, allergies and chronic obstructive pulmonary disease.

16. A method as claimed in claim 15 wherein said skin ailments include dermatitis, eczema and scleroderma.

17. A method as claimed in claim 15 wherein said immunological disorders include auto immune disorders such as plaque psoriasis, gutatte psoriasis, pustular psoriasis, psoriasis of the nails, psoriatic arthritis, rheumatoid arthritis, Crohn's disease, multiple sclerosis, irritable bowel disease, ankylosing spondilitis, systemic lupus erythremetosus and Sjogren's syndrome.

18. A method according to claim 15 wherein said allergies comprise asthma.

19. A herbal composition according to claim 1 wherein said interesting immunological and pharmacological properties, exhibited by the extract of the leaves and stem of Argemone mexicana plant, include immunosuppresion, lymphoproliferation inhibition, cytokine modulation, keratinocyte proliferation inhibition, keratolytic activity, endothelial cell proliferation inhibition, inhibition of cell adhesion molecule expression, phosphodiesterase (III, IV and V) inhibition, MEST inhibition, enzymes inhibition; cytokine expression inhibition, angiogenesis inhibition, proliferation marker inhibition, integrin inhibition, immune cell trafficking, lymphocyte trafficking, neutrophils trafficking, monocyte trafficking, and Th1 cytokine overexpression.

20. A herbal composition according to claim 19 wherein said cytokine modulation is IL-2 inhibition.

21. A herbal composition according to claim 19 wherein said cytokine modulation is IFN gamma inhibition.

22. A herbal composition according to claim 19 wherein said cytokine modulation is IL-10 induction.

23. A herbal composition according to claim 19 wherein said inhibition of cell adhesion molecule expression is ICAM-1.

24. A herbal composition according to claim 19 wherein said enzymes inhibition is p60src Tyrosine kinase and 5-Lipoxygenase inhibition.

25. The method of treatment of psoriasis and related biochemical and immunological disorders according to claim 3 which comprises oral administration to a mammal a herbal composition of claim 1, containing 10 to 50% by weight of the extract of the leaves and/or stem of Argemone mexicana

26. The method of treatment of psoriasis and related biochemical and immunological disorders according to claim 3 which comprises oral administration to a mammal a herbal composition of claim 1, wherein the amount of the extract of the leaves and/or stem of Argemone mexicana plant comprises from 50 to 5000 mg by weight of the herbal composition.

27. The method of treatment of psoriasis and related biochemical and immunological disorders according to claim 4 which comprises oral administration to a mammal a herbal composition of claim 2, comprising of from 10 to 50% by weight of an extract from the leaves and/or stem of Argemone mexicana plant and from 60 to 90% by weight of an extract from the fruits of Cuminum cyminum plant, the balance, if any, consisting of pharmaceutically acceptable carriers.

28. The method of treatment of psoriasis and related biochemical and immunological disorders according to claim 4 which comprises oral administration to a mammal a herbal composition of claim 2, wherein the amount of the extract of the leaves and/or stem of Argemone mexicana plant, comprises from 2 mg to 100 mg by weight of the herbal composition, the balance consisting of the extract from the fruits of Cuminum cyminum plant and pharmaceutically acceptable carriers.

29. The method of treatment of psoriasis and related biochemical and immunological disorders according to claim 3 which comprises topical application to a mammal a herbal composition of claim 1, wherein the amount of the extract of the leaves and/or stem of Argemone mexicana plant, comprises from 0.5% to 10% by weight of the extract, the balance, if any, consisting of pharmaceutically acceptable carriers.

30. The herbal composition according to claim 1 wherein said composition is in the form of tablets, capsules, syrups, elixirs and suspensions.

31. The herbal composition according to claim 2 wherein said composition is in the form of form of tablets, capsules, syrups, elixirs and suspensions.

32. The herbal composition according to claim 1 wherein said composition is in the form ointments, creams, lotions, oils or transdermal drug delivery systems.

33. The herbal composition according to claim 2 wherein said composition is in the form ointments, creams, lotions, oils or transdermal drug delivery systems.

34. The herbal composition according to claim 1 wherein said pharmaceutically acceptable carriers are selected from sugars such as lactose, sucrose, mannitol, sorbitol and xylitol; starches such as corn starch, tapioca starch and potato starch; hydroxypropylmethyl cellulose, carbomer; white wax, canauba wax; anionic emulsifying wax; white pertloatum; cellulose and its derivatives such as sodium carboxymethyl cellulose, ethyl cellulose and methyl cellulose; calcium phosphates such as dicalcium phosphate and tricalcium phosphate; sodium sulphate; calcium sulphate, polyvinylpyrrolidone, polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; beta-cyclodextrin; fatty alcohols and hydrolysed cereal solids; non-toxic compatible fillers; binders; disintegrants; buffers; preservatives; antioxidants; lubricants; and flavouring agents.

35. A herbal composition, for oral administration and topical application for the treatment of psoriasis and related biochemical and immunological disorders, comprising an effective amount of, a fraction, containing organic constituents, obtained from the leaves and/or stem of Argemone mexicana plant, in combination with pharmaceutically acceptable carriers, the said fraction exhibiting interesting immunological and pharmacological properties.

36. A method of treatment of psoriasis and related biochemical and immunological disorders comprising oral administration or topical application to a mammal the herbal composition of claim 35.

37. A herbal composition according to claim 35, wherein the fraction obtained from the extract of the leaves and/or stem of Argemone mexicana plant is a n-butanol-soluble fraction.

38. A herbal composition according to claim 35, wherein the fraction obtained from the extract of the leaves and/or stem of Argemone mexicana plant is a methanol-soluble fraction.

39. A herbal composition according to claim 35, wherein the fraction obtained from the extract of the leaves and/or stem of Argemone mexicana plant is a methanol-insoluble fraction.

40. A herbal composition according to claim 35, wherein the organic constituents present in the n-butanol-soluble fraction obtained from the extract of the leaves and/or stem of Argemone mexicana plant include alkaloids, flavonoids and other low molecular weight compounds.

41. A herbal composition according to claim 35, wherein the organic constituents present in the methanol-soluble fraction obtained from the extract of the leaves and/or stem of Argemone mexicana plant include amino acids, organic acids and salts.

42. A herbal composition according claim 35, wherein the organic constituents present in the methanol-insoluble fraction obtained from the extract of the leaves and/or stem of Argemone mexicana plant include free sugars/glycosides, organic acids and salts.

43. A herbal composition according to claim 35, wherein the fractions obtained from the extract of the leaves and/or stem of Argemone mexicana plant is a water extract.

44. A herbal composition according to any one of claim 35 wherein the fractions obtained from the extract of the leaves and/or stem of Argemone mexicana plant is a dry powder or viscous mass.

45. A herbal composition according to claim 35 wherein the amount of the n-butanol-soluble fraction, obtained from the leaves and/or stem of Argemone mexicana plant, comprises from 5 mg to 200 mg.

46. A herbal composition according to claim 35 wherein the amount of the methanol-soluble fraction, obtained from the leaves and/or stem of Argemone mexicana plant, comprises from 25 mg to 2550 mg.

47. A herbal composition according to claim 35 wherein the amount of the methanol-insoluble fraction, obtained from the leaves and/or stem of Argemone mexicana plant, comprises from 5 mg to 1250 mg.

48. The method of treatment of psoriasis and related biochemical and immunological disorders according to claim 36 which comprises oral administration to a mammal a herbal composition containing 5 mg to 200 mg of the n-butanol soluble fraction obtained from extract of the leaves and/or stem of Argemone mexicana plant, the balance consisting of pharmaceutically acceptable carriers.

49. The method of treatment of treatment of psoriasis and related biochemical and immunological disorders according to claim 36 which comprises oral administration to a mammal a herbal composition containing 25 to 2550 mg of the methanol-soluble fraction obtained from extract of the leaves and/or stem of Argemone mexicana plant, the balance consisting of pharmaceutically acceptable carriers.

50. The method of treatment of treatment of psoriasis and related biochemical and immunological disorders according to claim 36 which comprises oral administration to a mammal a herbal composition containing 5 to 1250 mg of the methanol-insoluble fraction obtained from extract of the leaves and/or stem of Argemone mexicana plant, the balance consisting of pharmaceutically acceptable carriers.

51. The herbal composition according claims 35 suitable for oral administration in the form of tablets, capsules, syrups, elixirs or suspensions.

52 The herbal composition according to claim 35, suitable for topical application, in the from of ointments, creams, gels, lotions, oils, or transdermal drug delivery systems.

53 The herbal composition according to claim 1 wherein said pharmaceutically acceptable carriers are selected from sugars such as lactose, sucrose, mannitol, sorbitol and xylitol; starches such as corn starch, tapioca starch and potato starch; hydroxypropylmethyl cellulose, carbomer; white wax, canauba wax; anionic emulsifying wax; white pertloatum; cellulose and its derivatives such as sodium carboxymethyl cellulose, ethyl cellulose and methyl cellulose; calcium phosphates such as dicalcium phosphate and tricalcium phosphate; sodium sulphate; calcium sulphate, polyvinylpyrrolidone, polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; beta-cyclodextrin; fatty alcohols and hydrolysed cereal solids; non-toxic compatible fillers; binders; disintegrants; buffers; preservatives; antioxidants; lubricants; and flavouring agents.

54 The herbal composition according to claim 2 wherein said pharmaceutically acceptable carriers are selected from sugars such as lactose, sucrose, mannitol, sorbitol and xylitol; starches such as corn starch, tapioca starch and potato starch; hydroxypropylmethyl cellulose, carbomer; white wax, canauba wax; anionic emulsifying wax; white pertloatum; cellulose and its derivatives such as sodium carboxymethyl cellulose, ethyl cellulose and methyl cellulose; calcium phosphates such as dicalcium phosphate and tricalcium phosphate; sodium sulphate; calcium sulphate, polyvinylpyrrolidone, polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; beta-cyclodextrin; fatty alcohols and hydrolysed cereal solids; non-toxic compatible fillers; binders; disintegrants; buffers; preservatives; antioxidants; lubricants; and flavouring agents.

55 The process for preparation of an extract of the leaves and/or stem of Argemone mexicana plant of any one of claims 1 and 2, comprising the steps of,
a) washing the leaves or stem of the plant with water,
b) grinding the leaves or stem of the plant with a solvent to give a coarse paste of the leaves or stem,
c) successively allowing the coarse paste to macerate or percolate in the presence of the solvent at room temperature for 16 hours,
d) successively extracting the organic constituents present in the leaves and stem of the plant with the solvent,
e) combining all the solvent extracts,
f) filtration of the combined solvent extracts,
g) centrifuging the filtrate obtained in step (f),
h) concentration of the filtrate to one fifth its volume, and
i) optionally, lyophilization or spray drying of the concentrate obtained from step
(h) to give a dry powder.

56. A process according to claim 55, wherein the solvent is selected from water, ethanol or mixtures thereof.

57. A process according to claim 55, wherein the solvent is evaporated in a rotary evaporator at a temperature below 50° C.

58. A process according to claim 55, wherein the organic constituents extracted include alkaloids, flavonoids, amino acids, organic acids, sugars/glycosides and salts.

59. A process according to claim 55, wherein the base number of the extract of the leaves and/or stem of Argemone mexicana plant is from 280 to 345.

60. A process for preparation of a fraction of the water extract of the leaves and/or stem of Argemone mexicana plant according to claims 35 comprising subjecting the said water extract to liquid-liquid partition chromatography.

61. A process for preparation of the n-butanol-soluble fraction of the leaves and/or stem of Argemone mexicana plant according to claims 37 comprising the steps of,
a. mixing the water extract of the leaves and/or stem of Argemone mexicana plant with n-butanol at room temperature, allowing the mixture to stand and separation of the n-butanol layer,
b. repetition of the process of step (a) twice,
c. keeping aside the separated aqueous layer,
d. combining all the three separated n-butanol extracts, containing organic constituents,
e. washing the combined n-butanol extracts with water, and
f. obtaining a viscous mass of the n-butanol-soluble fraction comprising evaporation, lyophilization or spray drying of the extract of step (d).

62. A process according to claim 61 wherein the solvent in step (f) is evaporated in a rotary evaporator below 50° C.

63. A process for preparation of the methanol-soluble fraction of the leaves and/or stem of Argemone mexicana plant according to claims 38 comprising the steps of,
a. mixing the water extract of the leaves and/or stem of Argemone mexicana plant with n-butanol at room temperature, allowing the mixture to stand and separation of the n-butanol layer,
b. repetition of the process of step (a) twice,
c. keeping aside the separated aqueous layer,
d. mixing the separated aqueous layer from step (c) with 6 to 7 times its volume of methanol,
e. centrifuging the precipitated solid, and
f. obtaining a dry powder of the methanol-soluble fraction comprising evaporation, lyophilization or spray drying of the supernatent solution of step (b).

64. A process according to claim 63 wherein the solvent is evaporated in a rotary evaporator below 50° C.

65. A process according to claim 63, wherein the base number of the methanol-soluble fraction is between 290-340.

66. A process for preparation of the methanol-insoluble fraction of the leaves and/or stem of Argemone mexicana plant according to claims 39 comprising the steps of,
a. mixing the water extract of the leaves and/or stem of Argemone mexicana plant with n-butanol at room temperature, allowing the mixture to stand and separation of the n-butanol layer,
b. repetition of the process of step (a) twice,
c. dissolving the methanol-insoluble solids obtained from step (b) of claim 73,
d. sonication of the solution of step (a),
e. centrifuging of the sonicated solution of step (b), and
f. obtaining a dry powder of the methanol-insoluble fraction comprising evaporation, lyophilization or spray drying of the solution of step (c).

67. A process according to claim 66 wherein the solvent is evaporated in a rotary evaporator below 50° C.

68. A process according to claim 66, wherein the base number of the methanol-insoluble fraction is between 350-380.
